SEQUENCE LISTING (4 sequences)

SEQ ID NO: 1 - DNA, 81 bp, Artificial Sequence
  Sequence is synthesized
  misc_feature (24)..(26): n is a, c, g, or t
  misc_feature (26)..(37): The nucleotides can  either be present or absent
  misc_feature (29)..(29): n is a, c, g, or t
  misc_feature (32)..(32): n is a, c, g, or t
  misc_feature (35)..(35): n is a, c, g, or t
  misc_feature (38)..(49): n is a, c, g, or t
  misc_feature (42)..(49): The nucleotides can  either be present or absent
  modified_base (80)..(81): 3′ phosphorothioated nucleotide
  misc_feature (81)..(81): n is a, c, g, or t
gggagttctacagtccgacgatcnnnbbnbbnbbnbbnnnnnnnnnnnnttttttttttttttttttttttttttttttvn

SEQ ID NO: 2 - DNA, 36 bp, Artificial Sequence
  Sequence is synthesized
  misc_feature (23)..(27): The nucleotides can  either be present or absent
  misc_feature (23)..(33): n is a, c, g, or t
  modified_base (34)..(36): 3′ phosphorothioated nucleotide
  misc_feature (35)..(36): The nucleotides can  either be present or absent
gccttggcacccgagaattccannnnnnnnnnnbbb

SEQ ID NO: 3 - DNA, 50 bp, Artificial Sequence
  Sequence is synthesized
  modified_base (49)..(50): 3′ phosphorothioated nucleotide
aatgatacggcgaccaccgagatctacacgttcagagttctacagtccga

SEQ ID NO: 4 - DNA, 63 bp, Artificial Sequence
  Sequence is synthesized
  misc_feature (25)..(30): n is a, c, g, or t
  modified_base (62)..(63): 3′ phosphorothioated nucleotide
caagcagaagacggcatacgagatnnnnnngtgactggagttccttggcacccgagaattcca

(19) United States
(12) Patent Application Publication
(10) Pub. No.: US 2023/0104375 A1
(43) Pub. Date: Apr. 6, 2023

(54) METHOD FOR MULTIPLEXABLE STRAND-SPECIFIC 3' END SEQUENCING OF MRNA TRANSCRIPTOME PRIMER SET, KIT AND APPLICATION THEREOF

(71) Applicants: Sequmed Biotechnology, Inc., Guangzhou (CN); Department of Biochemistry and Molecular Biology, Southern Medical University, Guangzhou (CN)

(72) Inventors: Xinghua PAN, Guangzhou (CN); Liyao MAI, Guangzhou (CN); Caiming CHEN, Guangzhou (CN); Yinbing QIU, Guangzhou (CN); Zhiwei LIAN, Guangzhou (CN)

(21) Appl. No.: 17/492,346

(22) Filed: Oct. 1, 2021

(51) Int. Cl. C12Q 1/6876; C12Q 1/6869

(52) U.S. Cl. CPC C12Q 1/6876; C12Q 1/6869; C12Q 2600/16

(57) ABSTRACT

The invention discloses a primer set and a multiplexable library building scheme for constructing an RNA sequencing library with a related reagent kit and an application thereof. The invention also discloses a corresponding data analysis method and a related instrument. The primer set used for RNA sequencing library construction includes the reverse transcription primers containing poly-dT, the 2nd strand cDNA primers particularly contain a random or semi-random portion at their 3′ end for universal initiation of the synthesis at multiple sites, or sequence capturing the 1^{st} strand of a specific cDNA, as well as their corresponding PCR primer 1 and 2. By this method, the library construction process is simple and the operation is convenient; the time of building the database was significantly shortened; it can carry out a large number of samples in a single run; the analysis process is simpler; the cost of database building, sequencing and analysis is significantly reduced.

### INCORPORATION-BY-REFERENCE OF SEQUENCE LISTING OR TABLE

The material in the accompanying sequence listing is hereby incorporated by reference in its entirety into this application. The accompanying file, named “151VP_001USU_Sequence_Listing_ST25.txt” was created on Sep. 28, 2021 and is 2.72 KB.

### TECHNICAL FIELD

The present disclosure relates to the field of RNA sequencing, and more particularly relates to an efficient method for multiplex RNA sequencing.

### BACKGROUND

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference is individually incorporated by reference. In terms of notation, hereinafter, [n] represents the nth reference cited in the reference list. For example, [1] represents the first reference cited in the reference list, namely, GMSA, “GSMA intelligence,” https://www.gsmaintelligence.com/, Accessed On: February 2019.

Ribonucleic acid sequencing (RNA-Seq) [1] (i.e. mRNA-seq) provides a digital molecular map of a biological sample, greatly facilitating the understanding of the mechanisms underlying health and pathogenic life processes [2-4]. In recent years, transcriptome sequencing has become a major application in next generation sequencing (NGS). Compared to microarray-based methods [5-7], RNA-Seq provides advantages such as higher signal-to-noise ratio, higher sensitivity, and lower batch effect and thus has become an essential tool in transcriptome profiling. Beyond quantifying gene expression, the data generated by full length RNA-Seq facilitates the discovery of novel transcripts [8,9], identification of alternatively spliced transcripts [10], detection of single nucleotide polymorphism [11] as well as allele-specific expression [12]. Following library construction, Illumina HiSeq sequencing platform is widely used for NGS. Other platforms are optional: Illumina NextSeq, MiniSeq, MiSeq, NovaSeq, HiSeq X10 and BGI MGISEQ.

A number of methods for RNA-Seq are available with procedures mostly distinguished in their library construction process. Currently, the major library construction methods of RNA-Seq for bulk cells including these from Illumina[13-17], NEB[18, 19], Qiagen and Invitrogen. The Illumina, NEB and Qiagen methods are similar, and typically include the following steps (as shown in as shown in FIG. 26 and FIG. 1A): 1) extract total RNA; 2) enrich the mRNA with oligo-dT magnetic beads; 3) fragmentize and reverse transcribe mRNA with random primers; 4) add an adenine deoxyribonucleotide (dA) to the 3′ end of the cDNA fragment to enable the ligation of the “Y”-shaped linker containing sample index and the sequencing primer binding site; 5) amplify the ligated cDNA by polymerase chain reaction (PCR) to obtain a library ready for sequencing. On the other hand, the Invitrogen method is partly different from the three providers above, and its main steps are as follows (as shown in FIG. 1B): 1) extract total RNA; 2) remove rRNA; 3) enrich mRNA; 4) fragmentize mRNA; 5) add a mixture of DNA/RNA linker, which consists of helper adaptors separately for 3′ and 5′ end; 6) initiate reverse transcription with random primers, and synthesize the first strand of cDNA; 7) amplify cDNA with P7 primer containing sample index and P5 primer to generate library ready for sequencing. Alternatively, template-switch-based approach in Smart-seq[20] joins a specific sequence tag at the 5′ end of RNAs, taking the advantage of the untemplated poly-dC-overhang added to full-length transcripts in reverse transcription, which is mostly used for RNA-seq for single cells and low quantity of cells.

In spite of successful application, the most common disadvantage with these conventional methods is that there are too many steps in library construction, which make them time-consuming and inefficient, especially when processing a large number of samples: each single sample needs to be operated separately, and also a relatively deep NGS is required to cover all coding sequences of the transcripts. Additionally, the aforementioned processes may be deficient in that the procedure for each protocol requires complicated steps for enabling the two ends of the cDNA to be targeted by the PCR primers for amplification.

On the other hand, these methods [16] generally end up getting data of full-length mRNA, which reflect a full spectrum of information for mRNA transcriptome, not only the quantitative expression message of all genes, but also the splicing isoforms, alternative expression and single nucleotide mutation information. However, in many cases of basic studies and clinical applications, it is desired only the differentially expressed genes (DEGs), while other information potentially useful are practically unused, hence resulting in a waste of resources and costs.

Therefore, a heretofore unaddressed need exists in the art to address the aforementioned deficiencies and inadequacies.

### SUMMARY

MustSeq, a new approach for transcriptome sequencing for population of cells, is described herein. The principles and features of MustSeq are fully described, with a comprehensive comparison with the most widespread RNA-seq methods: the golden standard TruSeq, and an alternation NEBNext RNA-seq, as well as QuantSeq—a 3′ end RNA-seq, by applying them to multiple cell lines and mouse livers. The feasibility, reproducibility, reliability, sequencing coverage and distribution, the detection of number of genes, lncRNAs, GO terms and KEGG pathways, as well as the efficiency of MustSeq are all sufficiently qualified. Although for genes, GO terms produced by a single test of MustSeq is relatively less than TruSeq, the measurements become at least in par with the TruSeq when triplicate tests are applied for MustSeq, attributing to its much higher efficience in labor and cost.

MustSeq may be used to efficiently carry out a medium to high-throughput analysis for a large number of samples to resolve practical scientific problems. MustSeq may also be applicable to certain types of formalin-fixed paraffin-embedded (FFPE) samples, and to expression-profiling of a panel of specific genes. The application of MustSeq in single-cell RNA sequencing could potentially become another powerful tool.

In the present embodiment, a method for performing ribonucleic acid (RNA) sequencing, is described, the method including the following steps: capturing 3′ ends of messenger RNA (mRNA) via a reverse transcriptase (RT) primer; performing reverse transcription on the mRNA, forming the first strand of complementary deoxyribonucleic acid (cDNAs); removing the mRNA from the first strand cDNA using ribonuclease (RNase) and heating-assisted disassociation; synthesizing (usually multiple, starting from different sites) second strand of cDNAs complementary to the first strand of cDNAs; pooling the multiple samples each with usually multiple second strands and the first strand of cDNAs; adding a P5 attachment site and a P7 attachment site and index to the second strands and the first strands of cDNA, meanwhile amplifying cDNA that comprise the one or more second strands and the first strand, via polymerase chain reaction (PCR); performing size selection on the amplified cDNA using an E-gel, magnetic beads, or HPLC, resulting in a sequencing-ready library; and performing paired-end sequencing.

In another embodiment, the RT primer includes an oligo-dT, a sample barcode, a unique molecular identifier (UMI), and a 5′ adapter.

In another embodiment, the oligo-dT, located in the 3′ end of the RT primer, is configured to capture a poly-A tail of the 3′ ends of mRNA.

In another embodiment, the UMI is the order of (NBB)ₙ, where N is adenine, thymine, cytosine, or guanine and B is thymine, cytosine, or guanine.

In another embodiment, the 5′ adapter is an amplification anchor for 5′ sequencing elements to be added to the sequencing library.

In another embodiment, the first 1-3 bases at both 5′ and 3′ end of RT primer are thiophosphate modified, to prevent degradation.

In another embodiment, the synthesizing is performed by using a chimeric primer and Klenow Fragment.

In another embodiment, the chimeric primer includes two deoxythymidine triphosphate (dTTP) nucleosides, six base pairs of random deoxynucleoside triphosphates (dNTP), and a 3′ adapter.

In another embodiment, the Klenow Fragment are configured to synthesize the second strand of cDNA using strand displacement and the Klenow Fragment lack both 3′ to 5′ exonuclease activity and 5′ to 3′ exonuclease activity.

In another embodiment, a P5 primer is used to add the P5 attachment site at the 5′ adapter and a P7 primer is used to add the P7 attachment site and the primer at the 3′ adapter.

In another embodiment, the E-gel is used to select the amplified cDNA fragment within the range from 100 to 900 base pairs to be included in the sequencing library.

In another embodiment, the capturing includes: diluting sample RNA; producing a sample by adding the sample RNA into a PCR tube containing the RT primer, a deoxynucleoside triphosphate (dNTP) mix, and nuclease-free water; vortexing the PCR tube, spinning down the PCR tube, placing the PCR tube on ice, incubating the PCR tube, and returning the PCR tube to ice; and spinning down the PCR tube and placing the PCR tube on ice.

In another embodiment, the performing reverse transcription includes: adding premixture, reverse transcriptase, Dithiothreitol (DTT), and RNase inhibitor to the PCR tube; incubating the PCR tube via a thermal cycler with a heated lid; holding the PCR tube at 4° C.; and placing the PCR tube on ice.

In another embodiment, the lid of thermal cycler is set to 60° C. and the PCR tube is incubated as follows: 45° C. for 10 minutes; then 50° C. for 10 minutes; then 55° C. for 10 minutes, and then 80° C. for 10 minutes.

In another embodiment, the removing includes: adding RNase mixture and nuclease-free water to the PCR tube; incubating the PCR tube at 37° C. for 20 minutes; incubating the PCR tube at 98° C. for 10 minutes; moving the PCR tube to room temperature; purifying and concentrating the sample; and eluting the sample in water.

In another embodiment, the RNase includes RNase A.

In another embodiment, the synthesizing includes: heating the PCR tube; adding a TTRanP primer to the PCR tube; cooling the PCR tube to room temperature for 3 minutes; placing the PCR tube on ice; adding Klenow Fragment, a Klenow fragment buffer, deoxynucleoside triphosphate (dNTP), and nuclease-free water to the PCR tube; and incubating the PCR tube with the thermal cycler.

In another embodiment, the amplifying includes: purifying the sample; adding PCR mix to the PCR tube, the PCR mix comprising an RP1 primer, an index primer, and PCR Master Mix; and performing PCR, wherein the performing PCR comprises the following steps: denaturing the sample; running thermal cycles on the sample; elongating the sample; and purifying the sample.

In another embodiment, the performing size selection includes: preparing the E-Gel; loading the sample into the E-Gel with a 50 base pair DNA ladder; performing electrophoresis; excising DNA fragments within the range of 250 base pairs-1000 base pairs from the E-Gel or beads; transferring the DNA fragments into a microcentrifuge tube; recovering and purifying the DNA fragments; adding preheated nuclease-water to the microcentrifuge tube; centrifuging the microcentrifuge tube; and measuring the concentration of the DNA fragments.

In another embodiment, the sample RNA is diluted to 1 μg/μL and the sample is produced by adding 1 μL of the sample RNA into the PCR tube.

In another embodiment, during the synthesizing, the lid of thermal cycler is set to 80° C. and the PCR tube is incubated as follows: 16° C. for 5 minutes; then 20° C. for 5 minutes; then 37° C. for 30 minutes; then 65° C. for 10 minutes, and then 75° C. for 20 minutes.

In another embodiment, the running thermal cycles comprises heating the sample to 98° C. for 10 seconds, then 60° C. for 30 seconds, and then 72° C. for 30 seconds.

### BRIEF DESCRIPTION OF THE DRAWINGS

The accompanying drawings illustrate one or more embodiments of the present disclosure and, together with the written description, serve to explain the principles of the present disclosure, wherein:

FIGS. 1A-1B illustrate workflows of RNA library construction using different methods, wherein FIG. 1A shows RNA library construction using TruSeq (TruSeq Stranded mRNA Library Prep, Illumina, cat. no. RS-122-2101), NEBNext (NEBNext® Ultra™ Directional RNA Library Prep Kit for Illumina, NEB, cat. no. E7420) and QIAseq Stranded mRNA Select Kit (QIAseq, cat. no. 180773) and FIG. 1B shows RNA library construction using on Total RNA-Seq (Invitrogen, cat. no. 4479789), in accordance with an embodiment of the present disclosure;

FIG. 2 illustrates an exemplary workflow of RNA library construction using MustSeq, in accordance with an embodiment of the present disclosure;

FIG. 3 illustrates a flowchart of an exemplary method for performing RNA library construction using MustSeq, in accordance with an embodiment of the present disclosure;

FIG. 4A-4D illustrate exemplary charts showing distribution and reproducibility of MustSeq, wherein FIG. 4A shows per base sequence content of read 1 mapping to a genome for cell line K562, FIG. 4B shows per base sequence content of read 2 mapping to a genome for cell line K562, FIG. 4C shows a Pearson correlation between technical replicates of a cell line for Jurkat with RPM>0, and FIG. 4D shows reads mapping to exons, introns, transcription start sites (TSS), transcription ending sites (TES), for MustSeq, QuantSeq, and TruSeq, in accordance with an embodiment of the present disclosure;

FIGS. 5A-5C illustrate quantification of the transcriptome for K562 sequenced with MustSeq in comparison with TruSeq and QuantSeq, wherein FIG. 5A shows a Pearson correlation for MustSeq with RPM>0, FIG. 5B shows a Pearson correlation for TruSeq with RPKM>0.1, and FIG. 5C shows a Pearson correlation for QuantSeq with RPM>0, in accordance with an embodiment of the present disclosure;

FIGS. 6A-6D illustrate an exemplary comparison between MustSeq, QuantSeq, and TruSeq for number of genes, wherein FIG. 6A shows a bar graph comparing the number of genes for MustSeq, QuantSeq, and TruSeq, FIG. 6B shows a comparison between TruSeq and MustSeq, FIG. 6C shows a comparison between TruSeq and QuantSeq, and FIG. 6D shows a comparison between MustSeq and QuantSeq, in accordance with an embodiment of the present disclosure;

FIGS. 7A-7D illustrate an exemplary comparison between MustSeq, QuantSeq, and TruSeq for lncRNA detection, wherein FIG. 7A shows a bar graph comparison for lncRNA detection for MustSeq, QuantSeq, and TruSeq, FIG. 7B shows a comparison between TruSeq and MustSeq, FIG. 7C shows a comparison between Truseq and QuantSeq, and FIG. 7D shows a comparison between QuantSeq and MustSeq, in accordance with an embodiment of the present disclosure;

FIG. 8 illustrates an exemplary read coverage of gene body percentage for different cell lines of cancer, in accordance with an embodiment of the present disclosure;

FIG. 9 illustrates an exemplary read coverage in gene SDF4, in accordance with an embodiment of the present disclosure;

FIG. 10 illustrates a distribution of reads mapping to a genome, in accordance with an embodiment of the present disclosure;

FIGS. 11A-11B illustrate exemplary Pearson correlation coefficients of transcriptomic profiles sequenced with MustSeq with RPM>0, wherein FIG. 11A shows a Pearson correlation coefficient for HL60 and FIG. 11B shows a Pearson correlation coefficient for Hela, in accordance with an embodiment of the present disclosure;

FIG. 12 illustrates an exemplary heatmap of the top 20 different genes between Jrurkat and K562 when sequenced with MustSeq, where RPM>10, |log₂ FC|>2, and FDR<0.01, in accordance with an embodiment of the present disclosure;

FIG. 13 illustrates an exemplary volcano plot of different genes between K562 and Jurkat when sequenced with MustSeq, where RPM>10, |log₂ FC|>2, FDR<0.01, and n=2, in accordance with an embodiment of the present disclosure;

FIGS. 14A-14D illustrate a transcriptomic analysis of mouse hepatocarcinoma (HCC) and health liver, wherein FIG. 14A shows a comparison between NEBNext and MustSeq for the genes, FIG. 14B shows a comparison between MustSeq (Triplicate) and NEBNext for the genes, FIG. 14C shows a comparison between NEBNext and MustSeq for GO terms, and FIG. 14D shows a comparison between NEBNext and MustSeq for KEGG pathways, in accordance with an embodiment of the present disclosure;

FIGS. 15A-15B illustrate statistical analysis based on separated result of triplicates, wherein FIG. 15A shows the top 20 GO terms enriched in a healthy liver by MustSeq and FIG. 15B shows the top 20 GO terms enriched in a healthy liver by NEBNext, in accordance with an embodiment of the present disclosure;

FIG. 16 illustrates an exemplary heatmap of Pearson correlation coefficient between a healthy liver and HCC, in accordance with an embodiment of the present disclosure;

FIG. 17 illustrates an exemplary heatmap of the top 20 genes between a healthy liver and an HCC liver sequenced by MustSeq, where |log₂ FC|>2 and FDR<0.01, in accordance with an embodiment of the present disclosure;

FIG. 18 illustrates an exemplary volcano plot of different genes between an HCC liver and a healthy liver when sequenced by MustSeq, where RPM>0.1, |log₂ FC|>2, FDR<0.01, and n=3, in accordance with an embodiment of the present disclosure;

FIGS. 19A-19D illustrate exemplary graphs showing performance of MustSeq for a pool of total RNA from cell line K562, wherein FIG. 19A shows a total RNA integrity RIN (9.9), FIG. 19B shows a saturation curve of MustSeq reads compared with a number of genes detected, FIG. 19C shows a per base sequence quality of cell line Jurkat, and FIG. 19D shows a per sequence quality of cell line Jurkat, in accordance with an embodiment of the present disclosure;

FIGS. 20A-20L illustrate reads mapping to exon, intron, and intergenic regions for two replicates of four cell lines, wherein FIG. 20A shows reads using MustSeq for K562 repeat 1, FIG. 20B shows reads using MustSeq for K562 repeat 2, FIG. 20C shows reads using MustSeq for Jukrat repeat 1, FIG. 20D shows reads using MustSeq for Jukrat repeat 2, FIG. 20E shows reads using MustSeq for Hela repeat 1, FIG. 20F shows reads using MustSeq for Hela repeat 2, FIG. 20G shows reads using MustSeq for HL60 repeat 1, FIG. 20H shows reads using MustSeq for HL60 repeat 2, FIG. 20I shows reads using TruSeq for K562 repeat 1, FIG. 20J shows reads using TruSeq for K562 repeat 2, FIG. 20K shows reads using QuantSeq for K562 repeat 1, and FIG. 20L shows reads using QuantSeq for K562 repeat 2, in accordance with an embodiment of the present disclosure;

FIG. 21 illustrates an exemplary graph for reads mapping to exons, introns, TSS, and TES showing comparison between MustSeq, QuantSeq, and TruSeq sequencing results with RNA from cell like K562, in accordance with an embodiment of the present disclosure;

FIGS. 22A-22R illustrate exemplary comparisons for cell ine K562 between MustSeq, QuantSeq, and TruSeq, wherein FIGS. 22A-22C show comparison in genes, FIGS. 22D-22I show a comparison in lncRNAs, FIGS. 22J-22L show a comparison in GO terms, and FIGS. 22M-22R show a comparison in KEGG pathways, in accordance with an embodiment of the present disclosure;

FIGS. 23A-23C illustrate exemplary graphs showing a demonstration of read coverage by MustSeq compared with TruSeq, wherein FIG. 23A shows read coverage for Housekeeping gene RPL19, FIG. 23B shows read coverage of gene JUND, and FIG. 23C shows read coverage of gene CPTP, in accordance with an embodiment of the present disclosure;

FIGS. 24A-24F illustrate exemplary comparisons between MustSeq and NEBNext when applied on a mouse HCC liver and a mouse healthy liver, wherein FIG. 24A shows a comparison between NEBNext and MustSeq for gene detection on a HCC liver, FIG. 24B shows a comparison between NEBNext and MustSeq (Triplicate) for gene detection, FIG. 24C shows a comparison between NEBNext and MustSeq for lncRNA detection in a healthy liver, FIG. 24D shows a comparison between NEBNext and MustSeq for lncRNA detection in an HCC liver, FIG. 24E shows a comparison between NEBNext and MustSeq for enriched GO terms in an HCC liver, and FIG. 24F shows a comparison between NEBNext and MustSeq for enriched KEGG pathways, in accordance with an embodiment of the present disclosure;

FIGS. 25A-25B illustrates exemplary graphs for enriched GO terms, wherein FIG. 25A shows top 20 GO terms enriched by MustSeq for an HCC liver, and FIG. 25B shows top 20 GO terms enriched by NEBNext for an HCC liver, in accordance with an embodiment of the present disclosure;

FIG. 26 illustrates a table showing the methodology of MustSeq when compared to other full-length RNA sequencing methods for RNA library construction, in accordance with an embodiment of the present disclosure;

FIG. 27 illustrates barcode oligonucleotide, in accordance with an embodiment of the present disclosure;

FIG. 27 illustrates a table showing exemplary reverse transcription primers (BarcodedT24) with different barcodes for MustSeq, in accordance with an embodiment of the present disclosure;

FIG. 28 illustrates a table showing an exemplary distribution of reads mapping to a genome as a percentage, in accordance with an embodiment of the present disclosure; and

FIGS. 29 and 30 illustrates a table showing a procedural comparison between QuantSeq and MustSeq, in accordance with an embodiment of the present disclosure.

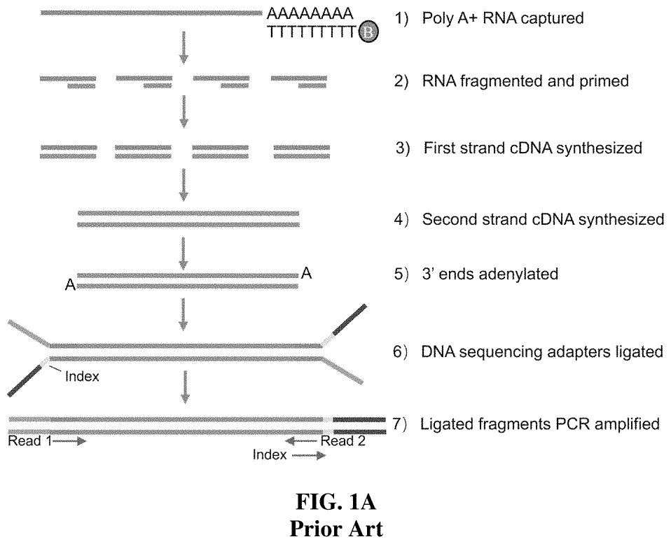

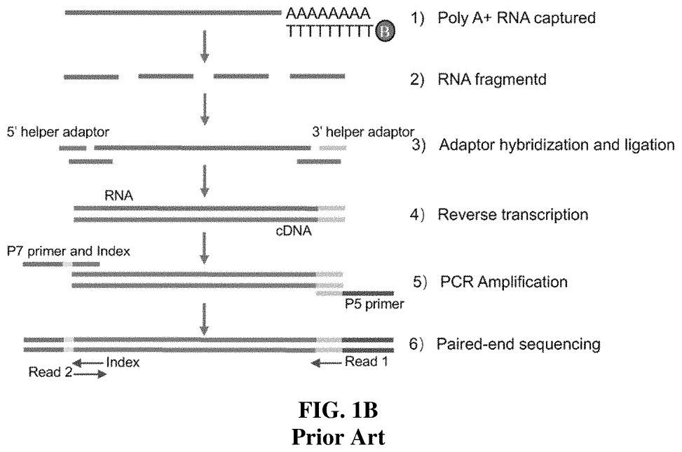

### DETAILED DESCRIPTION

The present disclosure will now be described more fully hereinafter with reference to the accompanying drawings, in which exemplary embodiments of the present disclosure are shown. The present disclosure may, however, be embodied in many different forms and should not be construed as limited to the embodiments set forth herein. Rather, these embodiments are provided so that this disclosure is thorough and complete, and will fully convey the scope of the present disclosure to those skilled in the art. Like reference numerals refer to like elements throughout.

The terms used in this specification generally have their ordinary meanings in the art, within the context of the present disclosure, and in the specific context where each term is used. Certain terms that are used to describe the present disclosure are discussed below, or elsewhere in the specification, to provide additional guidance to the practitioner regarding the description of the present disclosure. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It is appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the present disclosure or of any exemplified term. Likewise, the present disclosure is not limited to various embodiments given in this specification.

It is understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It is understood that, although the terms Firstly, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the present disclosure.

It is understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It is also appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the present disclosure. As used herein, the singular forms “a,” “an,” and “the” are intended to include the multiple forms as well, unless the context clearly indicates otherwise. It is further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It is understood that relative terms are intended to encompass different orientations of the device in addition to the orientation shown in the figures. For example, if the device in one of the figures is turned over, elements described as being on the “lower” side of other elements will then be oriented on the “upper” sides of the other elements. The exemplary term “lower” can, therefore, encompass both an orientation of lower and upper, depending on the particular orientation of the figure. Similarly, for the terms “horizontal”, “oblique” or “vertical”, in the absence of other clearly defined references, these terms are all relative to the ground. Similarly, if the device in one of the figures is turned over, elements described as “below” or “beneath” other elements will then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present disclosure belongs. It is further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around,” “about,” “substantially,” “generally” or “approximately” shall generally mean within 20 percent, preferably within 10 percent, and more preferably within 5 percent of a given value or range. Numerical quantities given herein are approximate, meaning that the terms “around,” “about,” “substantially,” “generally” or “approximately” can be inferred if not expressly stated.

As used herein, the terms “comprise” or “comprising,” “include” or “including,” “carry” or “carrying,” “has/have” or “having,” “contain” or “containing,” “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. It should be understood that one or more steps within a method may be executed in different order (or concurrently) without altering the principles of the present disclosure.

Embodiments of the present disclosure are illustrated in detail hereinafter with reference to accompanying drawings. It should be understood that specific embodiments described herein are merely intended to explain the present disclosure, but not intended to limit the present disclosure.

In order to further elaborate the technical means adopted by the present disclosure and its effect, the technical scheme of the present disclosure is further illustrated in connection with the drawings and through specific mode of execution, but the present disclosure is not limited to the scope of the implementation examples.

The present disclosure relates to the field of RNA sequencing, and more particularly relates to an efficient method for high-throughput RNA sequencing.

In response to the need for highly efficient generation of libraries for RNA-seq, a new alternative method for multiplexable, strand-specific, 3′ end mRNA-seq for bulk cells is described, termed MustSeq. MustSeq may be utilized to directly select and amplify the mRNA 3′ ends from the total RNA of one sample, or multiple populations of cells in a run, immediately ready for NGS. The present disclosure examines the reproducibility, efficiency, accuracy and other related features of the method in four cancer cell types. The results were compared with TruSeq, a classic library construction method for bulk RNA-seq, and QuantSeq [21, 22], an industrial product for 3′ end representative sequencing, using leukemia cell line K562. The present disclosure also applied both NEBNext and MustSeq on mouse liver cancer and demonstrated that MustSeq faithfully and efficiently revealed the DEG profiles. MustSeq may be used to append additional barcode tags to the reverse transcription primer, which were sequenced with each molecule and each sample, enabling pooling of multiple samples at a very early stage. MustSeq is a reliable, reproducible and efficient approach for bulk RNA-seq and potentially applicable to formalin-fixed paraffin-embedded (FFPE) samples and single cell analysis.

RNA-seq has been widely used to reveal the molecular mechanism of variants of life processes. The present disclosure describes an alternative method, MustSeq, which generates multiple second strands along a single 1^{st} strand cDNA by random-priming initiation, immediately after reverse transcription for each RNA extract using barcoded poly-dT primers, then 3′ ends-enriching PCR is applied to construct the library. Unlike conventional RNA-seq, MustSeq eliminates procedures such as mRNA isolation, fragmentation, and RNA 5′-end capture, enables early pooling of multiple samples, and requires only one twentieth of sequencing reads of full-length sequencing. The present disclosure demonstrates the power and features of MustSeq by comparison with TruSeq and NEBNext RNA-seq, two conventional full-length methods, and QuantSeq, an industrial 3′ end method. In cancer cell lines, the reads distribution of CDS-exon as well as genes, lncRNAs and GO terms detected by MustSeq are closer than QuantSeq to TruSeq. In mouse hepatocarcinoma and healthy livers, MustSeq enriches the same pathways as by NEBNext, and reveal the molecular profile of carcinogenesis. Overall MustSeq is a robust and accurate RNA-seq method providing efficient library construction, sequencing and analysis, particularly valuable for analysis of differentially expressed genes with a large number of samples. MustSeq will greatly accelerate the application of bulk RNA-seq on different fields, and potentially applicable for single cell RNA-seq.

Materials and Methods

Oligonucleotides

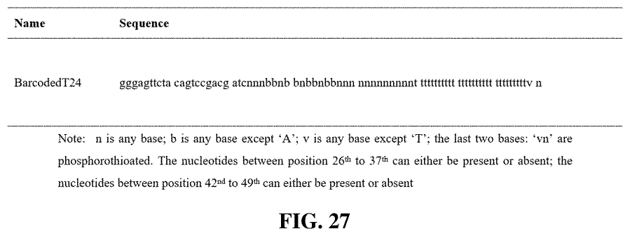

BarcodedT24 (as shown in FIG. 27) (5′-gggagttcta cagtccgacg atcnnnbbnb bnbbnbbnnn nnnnnnnnnt tttttttttt tttttttttt tttttttttv n-3′). This oligo may be used to capture the RNAs containing a poly-A tail. The middle of this oligonucleotide ‘NBB’ is the unique molecular identifier, where ‘n’ is any base, ‘b’ is any base except base ‘A’, ‘v’ is any base except ‘T’; the nucleotides between position 26th to 37th can either be present or absent; the nucleotides between position 42^{nd} to 49th can either be present or absent; the last two bases, ‘vn’ are phosphorothioated or not phosphorothioated.

TTRanP (5′-gccttggcac ccgagaattc cannnnnnnn nnnbbb-3′). This oligo may act as the primer in the second strand synthesis after reverse transcription (RT). ‘n’ is any base and ‘b’ is any base except base ‘A’; the nucleotides between position 23^{th} to 27^{th} can either be present or absent; the nucleotides between position 35^{rd} to 36^{th} can either be present or absent; the last three bases, ‘bbb’ are phosphorothioated.

RP1 (5′-aatgatacgg cgaccaccga gatctacacg ttcagagttc tacagtccga-3′); RPIX (5′-caagcagaag acggcatacg agatnnnnnn gtgactggag ttccttggca cccgagaatt cca). The primer RP1 may be used to capture the poly-dA/dT end of the cDNA during library amplification. The primer RPIX may be used to capture the TTRanP primer end during library amplification, of which the “X” indicates a primer with library index #X. RP1 and RPIX may be paired primers of the PCR.

All the oligonucleotides mentioned above were dissolved in a TE buffer, to a final concentration of 100 μM, and stored at −20° C. up to 6 months before use.

Primer:

First-strand reverse transcription primer, or oligo dT primer (5′-3′), wherein b is G, T or C, b represents G, A, or C, n represents A, T, C or G.

BarcodedT24:
(SEQ ID NO. 1)
gggagttctacagtccgacgatcnnnbbnbbnbbnbbnnnnn
nnnnnnnttttttttttttttttttttttttttttttvn
Second strand cDNA synthesis primer, or
random primer (5′-3′)
TTRan RTpri:
(SEQ ID NO. 2)
gccttggcac ccgagaattc cannnnnnnn nnnbbb

PCR primers for the first and second library amplification, referred to as amplification primers (5′-3′)

RPI (Compatible with cDNA first-strand primer, which is equivalent to the 3′primer for amplifying mRNA):

(SEQ ID NO.3)
aatgatacgg cgaccaccga gatctacacg
ttcagagttc tacagtccga

RPIX (Compatible with cDNA second-strand primers, equivalent to primers that amplify and capture mRNA in the far 3′end direction):

(SEQ ID NO. 4)
aagcagaag acggcatacg agatnnnnnn gtgactggag
ttccttggca cccgagaatt cca

Cell Lines and Mouse Tissues

Cell line K562 was prepared as described [23]. Cell line Hela, Jurkat and HL60 were prepared as described [24]. C57BL/6 mice were used to generate the hepatocarcinoma (HCC). The mice were anesthetized by pentobarbital injection, and then the liver was taken for downstream experiments after heart perfusion. Total RNA was extracted according to the manufacturer's instructions.

RNA isolation, first strand generation cDNA synthesis and sample barcoding

The total RNA may be extracted from a population of cells with RNeasy Micro Kit (Qiagen, cat. No. 74004). The RNA may be diluted to 1 μg/μl (or a different lower concentration as specified otherwise), and 1 μl of each sample may be added into a 0.2 ml PCR tube containing 10 μM BarcodedT24 primer, 10 nmol deoxynucleoside triphosphate (dNTP) mix (Thermo Fisher, cat. no. R0191) and nuclease-free water in total 4.5 μl volume. The same quantity of RNA is required for all samples to be multiplexed in library preparation or analyzed in parallel. The tubes may be vortexed quickly, spun down, and placed on ice, which may then be incubated at 72° C. for 3 minutes, and returned to ice. The tubes may then be spun down again to collect liquid at the bottom of the tubes and placed back into ice. In each tube a total of 5.5 μl premixture with 100 U Superscript IV reverse transcriptase (200 U/μl, Thermo Fisher, cat. no. 18090050), 0.05 μmol DTT (0.1 M, Thermo Fisher, cat. no. 18090050) and 12 U RNase Inhibitor (40 U/μl, Clontech, cat. no. 2313A) may be added. On a thermal cycler with a heated lid set to 60° C., the samples may then be incubated as follows: 45° C. for 10 minutes; then 50° C. for 10 minutes; then 55° C. for 10 minutes, and then 80° C. for 10 minutes. The tubes may be held at 4° C. before back on ice. Total volume 4.5 μl of mixture containing RNase mixture and nuclease-free water may then be added to fulfill a reaction volume of 15 μl for each RNA sample or sample pool, which may be incubated at 37° C. for 20 minutes, then 98° C. for 10 minutes, and finally moved to room temperature. For multiplex library construction, a set of samples after reverse transcription and RNA removed may be pooled. The reaction mixture may be purified and concentrated with 1× volume of AMPure XP Beads (Beckman coulter, cat. no. A63880), according to its manufacture, and eluted in 15 μl water.

The 3′ end preferred second strand synthesis

The primer TTRanP 1 μl×100 μM may be added in each tube, which was heated at 95° C. for 10 minutes. The tubes may be cooled to room temperature (−24° C.) for 3 minutes, then placed on ice. A mixture of 2.5 U Klenow fragment (5 U/μl, Takara, cat. no. 2140A), 1× of Klenow fragment buffer, 5 nmol dNTP mix and 1 μl of nuclease-free water may be added to each tube from the previous step. On a thermal cycler with a heated lid set to 80° C. the reaction mixtures may be incubated as follows: 16° C. for 5 minutes; then 20° C. for 5 minutes; then 37° C. for 30 minutes; then 65° C. for 10 minutes, and inactivated the enzyme at 75° C. for 20 minutes.

Enrichment and Amplification of the 3′ Ends by PCR

A 0.8× volume of AMPure XP Beads (Beckman coulter, cat. no. A63880) may be used to purify each reaction mixture or sample pool from the previous process. A volume of 27 μl of PCR mix containing 10 mol RP1, 10 mol RPIX (index primer) and 1× Phusion High-Fidelity PCR Master Mix (NEB, cat. no. M0531S) may be added to each tube, and PCR may be performed with this program: denaturation at 98° C. for 30 seconds, then running up to 28 thermal cycles (98° C. for 10 seconds, then 60° C. for 30 seconds, and then 72° C. for 30 seconds) for a single sample. For a collection of 10 samples in multiplex library generation, 18 cycles may be applied. Finally, elongation may be carried out at 72° C. for 10 minutes and held at 4° C. AMPure XP Beads 0.6× volume may be used to purify PCR products again.

Size Selection and Gel Recovery

The 2% E-Gel (Thermo Fisher, cat. no. G402002) may be prepared. The preliminary amplification product of library obtained above may be loaded onto the gel, with 50 bp DNA Ladder (Thermo Fisher, cat. no. 10488099). After electrophoresis (Thermo Fisher, cat. no. G8100), the DNA fragments (library) of 400 bp-1000 bp from the gel may be excised using a razor blade or other device and transferred into a new 1.5 ml microcentrifuge tube, and the DNA may be recovered and purified from the gel (ZYMO RESEARCH, cat. no. D4007). Finally, 8 μl 60° C. preheated nuclease-water may be added directly to the column matrix. The column may be placed into a 1.5 ml tube and centrifuged 30-60 seconds to elute DNA and the step may be repeated. Qubits (Thermo Fisher, cat. no. Q33238) may be used to measure the concentration of each library.

Other Methods for Library Construction for RNA-Seq

The QuantSeq 3′ mRNA-Seq Library Prep Kit for Illumina (in this report referred as QuantSeq, Lexogen cat. no. 015.24), TruSeq Stranded mRNA Library Prep (referred as TruSeq, Illumina, cat. no. RS-122-2101), NEBNext® Ultra™ Directional RNA Library Prep Kit for Illumina (referred as NEBNext, NEB cat. no. E7420) were applied to construct the libraries according to the manufacturer's instructions.

RNA Sequencing on NGS

Libraries were paired-end sequenced (150 bp×2) on Illumina HiSeq X10 instrument according to the manufacturer's procedure.

Expression Analysis Pipeline

MustSeq analysis pipeline included multiple steps. The data may be decoded by different sample barcodes. FastQC v0.11.8 was used to demonstrate data quality. Trimmomatic [25] v0.39 may be used to remove adapters and low-quality sequences, and the polyA of Read2 may be removed using several Perl scripts. For alignment, TopHat2 [26] v2.1.1 may be applied to map the clean reads to reference genome (the reference genome GRCh38 for Homo sapiens and GRCm38 for Mus musculus), and the annotation file may be obtained from the National Center for Biotechnology Information (NCBI). SAMtools [27] v1.11 may be applied to sort bam files after the mapping. FeatureCounts [28] v2.0.1 may be used to count the reads of sorted bam files. Reads per million mapped reads (RPM) (for MustSeq and QuantSeq) and Reads Per Kilobase per Million mapped reads (RPKM) (for TruSeq and NEBNext) may be calculated to normalize the results and build [29] an expression matrix for downstream statistical and functional analysis.

Results

Procedure of Library Construction for MustSeq

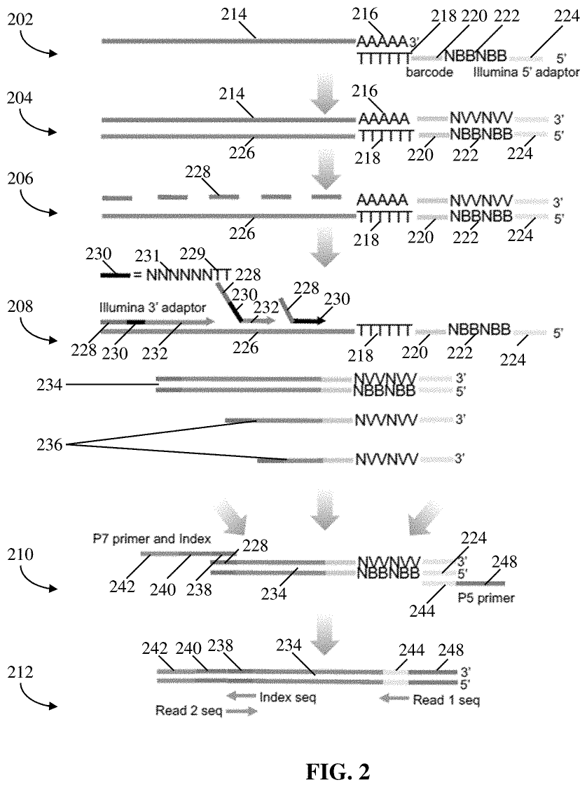

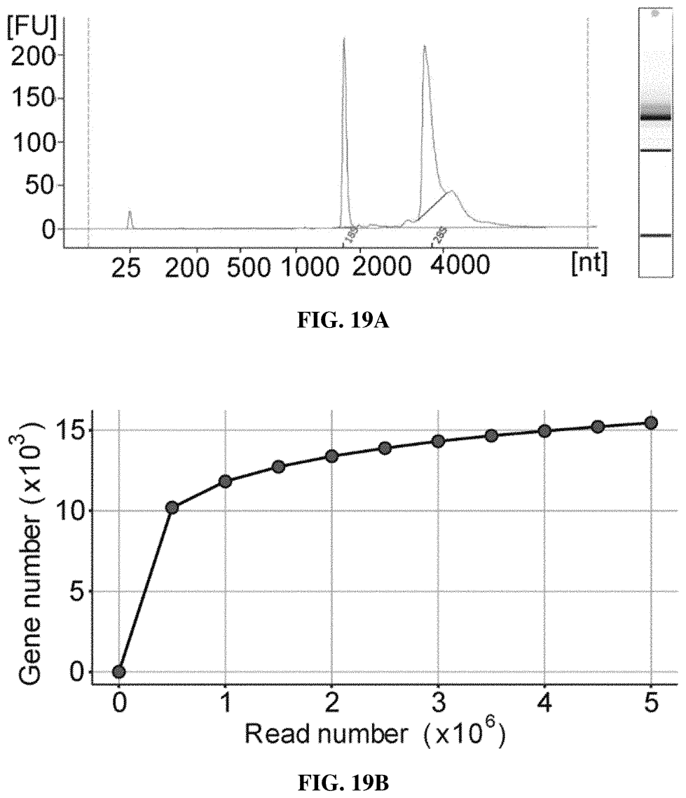

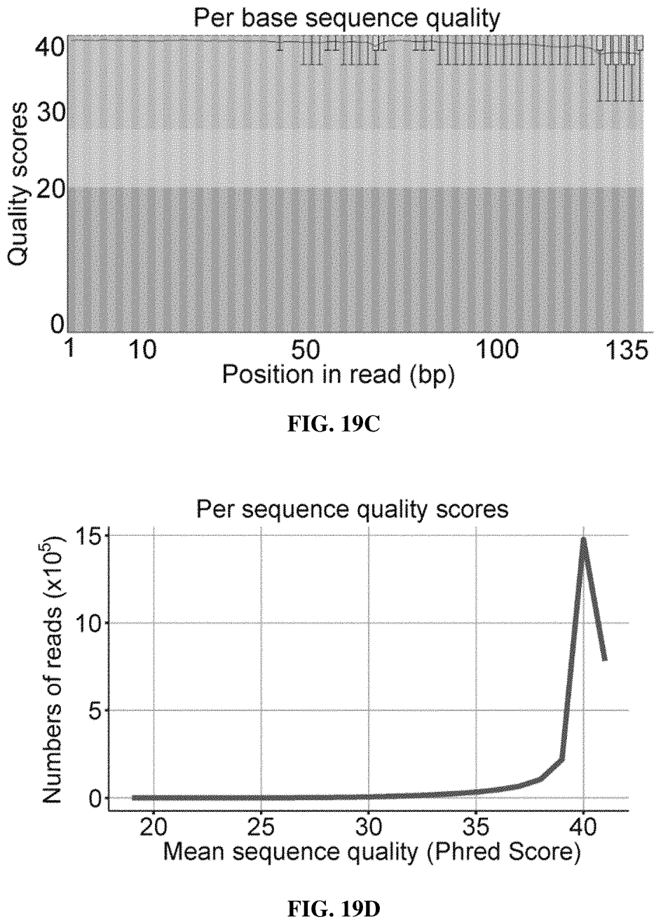

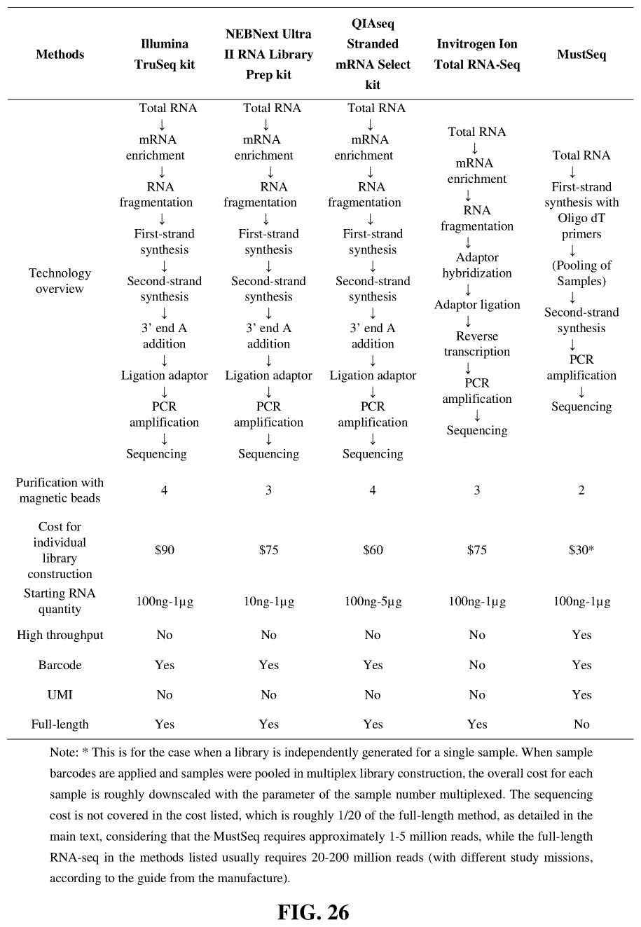

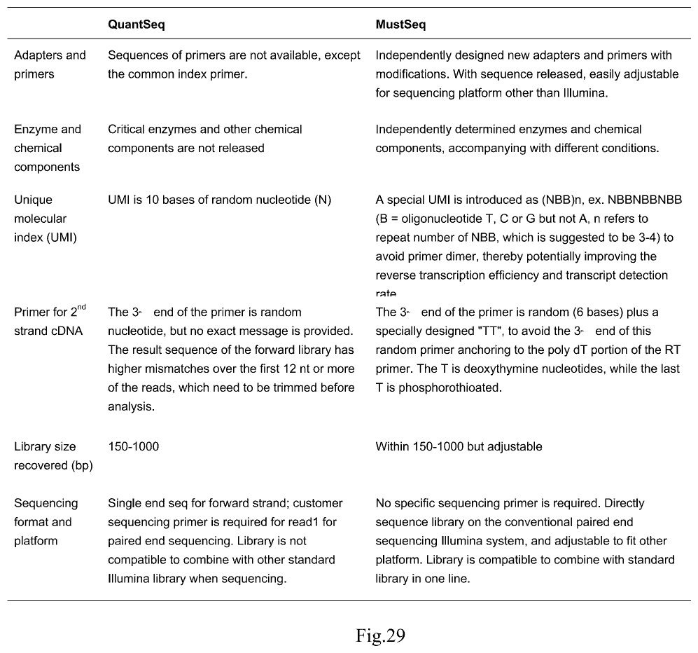

MustSeq is designed as an alternative short-cut method for library construction and analysis for the 3′ ends of mRNA transcriptome from total RNA (as shown in FIG. 2, FIG. 26, and FIG. 29). The first step (step 202 of FIG. 2) may be to capture the 3′ ends of mRNA 214 with a reverse transcription (RT) primer, which may be composed of oligo-dT 218, sample barcode 220, unique molecular identifier (UMI) 222 and Illumina-compatible 5′ adaptor 224. During reverse transcription, oligo-dT 218 plays the role of capturing 3′ poly-A tail 216 of mRNA 214 transcripts. Total RNA, RIN>7 (as shown in FIG. 19A), may be used as an input without extra step of removing rRNA required in some conventional methods. Sample barcode 220 and UMI 222 may serve as specific markers for multiple samples and mRNA molecules, respectively. Sample barcode 220 may be used to track each sample's identity and enable very early pooling of multiple samples into a single tube for the following procedures in library construction and sequencing, while UMI 222 may be used to aid in the correction of any potential distortion of operation bias in the sequencing data afterwards. Conventional UMI design is generally in the form of NNNNNN [16, 22, 30, 31] (where N=any nucleotide, and the number of N is variable), which occasionally constitutes multiple deoxyadenylic acids (poly-dA) and leads to formation of primer dimers with oligo-dT. In the present embodiment, UMI 222 with an order of (NBB)ₙ, (e.g., NBBNBBNBB, where B is thymine, cytosine, or guanine but not adenine, and n refers to 3 to 4 repeats) may be used to avoid the formation of primer dimers and for easy identification, thereby improving reverse transcription efficiency and DEG analysis accuracy. The outside (5′ end) of the RT primer described above may be Illumina 5′ adapter 224 used as an amplification anchor for 5′ Illumina sequencing elements to be added to the sequencing library.

RNase H may be used to hydrolyze mRNA 214 on first strand of complementary DNA (cDNA) 226 after reverse transcription (i.e., step 204), resulting in mRNA fragments 228 in step 206. After RNase mixture and heating-assisted dissociation and removal of mRNA fragments 228, second strand of cDNA 232 may be synthesized in step 208 using a chimeric primer with random or semi-random nucleotides at its 3′ end for priming second strand of cDNA 232 at multiple sites along first strand of cDNA 226, and with Illumina 3′ adaptor 228 at its 5′ end to anchor a PCR primer and to add the sequencing tag for 3′ end Illumina sequencing elements compatible to the NGS platform. The chimeric primer starting from its 3′ end includes, for example, without limitation, intermediate sequence 230 including two deoxythymidine triphosphate (dTT) nucleosides 229 (i.e., TT), 6-bp random dNTPs 231 (i.e., NNNNNN), and 3′ Illumina adapter 228. Klenow Fragment lacking both 3′->5′ exonuclease activity and 5′->3′ exonuclease activity may be used for second strand synthesis. As the Klenow Fragment used for MustSeq lack both 3′->5′ exonuclease activity and 5′->3′ exonuclease activity, digestion of the cDNA at the 3′ end is prevented. The Klenow Fragment possess strand displacement activity such that multiple second strands may be generated on each first strand template towards the 3′ terminal. For example, single stranded cDNA 236 and double stranded cDNA 234 of different lengths may be a result of second strand cDNA synthesis. Both single stranded cDNA 236 and double stranded cDNA 234 may be used in the subsequent steps, but for simplicity, only double stranded cDNA 234 is shown. To prevent the 3′ end of the chimeric primer anchoring to the poly-dT portion of the RT primer, 2 deoxythymine nucleotide (TT) may be used at the 3′ end of the chimeric primer. Thus, second strand synthesis of cDNA may be performed using the chimeric primers and the Klenow Fragment. Double stranded cDNA 234 and single stranded cDNA 236 may be pooled before starting amplification. With this design, the process of adapter ligation, polyadenylation, template switch and other laborious steps of the aforementioned conventional RNA sequencing processes may be avoided while a PCR tag, the 3′ Illumina sequencing tag at the opposite end of poly-dT/poly-dA of the amplicon, is built.

In a step 210, P5 attachment site 248 may be added via P5 primer 244 at Illumina 5′ adapter 224, P7 attachment site 242 and index 240 may be added via P7 primer 238 at Illumina 3′ adapter 228, the cDNA may be amplified via PCR, and size selection may be performed to select cDNA fragments with 400-1000 base pairs.

The P5 attachment site 248, index 240, and P7 attachment site 238 may be added during PCR. Initially, P5 primer 244 may be used to add P5 attachment site 248 at the 5′ end of double stranded cDNA 234 and an index sequencing primer and read 2 sequencing primer may be added to the 3′ end of double stranded cDNA 234. P7 primer 238 may then be used to add index 240 and P7 attachment site 242 to the 3′ end of double stranded cDNA 234.

The process for adding P5 attachment site 248, P7 attachment site 242, and index 240 may be similar for double stranded cDNA 234 and single stranded cDNA 236. Double stranded cDNA 234 may first be dissociated into two single strands of cDNA during a denaturation step of PCR, and within the first two cycles of PCR, P5 attachment site 248 and P7 attachment site 242 may be added to either end of the single stranded cDNA, and subsequent cycles of PCR further amplify the cDNA including P5 attachment site 248 and P7 attachment site 242 until a complete sequencing library is constructed.

Based on the 5′ and 3′ Illumina sequencing priming tags, PCR may be applied to select and amplify the 3′ end sequences of the mRNA transcriptome and build the complete sequencing library. Because of PCR-suppression effect [32], only the amplicons containing the 3′ end sequences of mRNAs are efficiently amplified and enriched, while any product that has only the 5′ Illumina sequencing tag on both ends of the amplicon is excluded.

In addition to the simplified procedure, the method of the present disclosure enables medium- or high-throughput construction of the RNA-seq library for multiple samples in one tube, and immediately after an independent pre-processing of each individual sample in different tubes. The individual treatment of each RNA sample results in the first strand (and optionally the second strand) synthesis. Additionally, labeling each RNA sample with the sample barcode during the reverse transcription allows for tens to hundreds of samples to be pooled in one tube for the following processes. An index may be applied on the final library to label the batch of samples. Thus, the design of the present embodiment greatly reduces the effort required to handle processing.

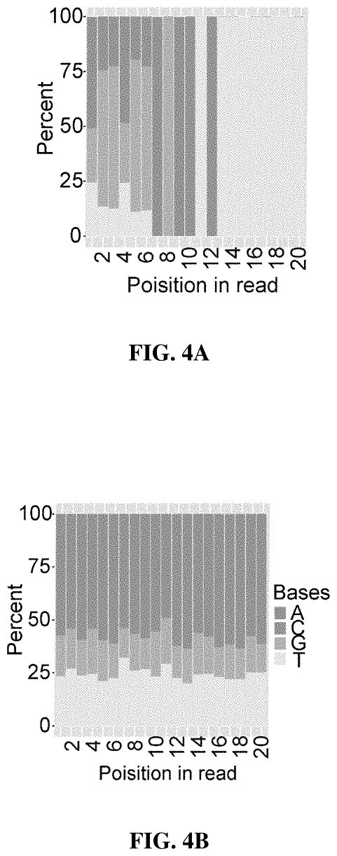

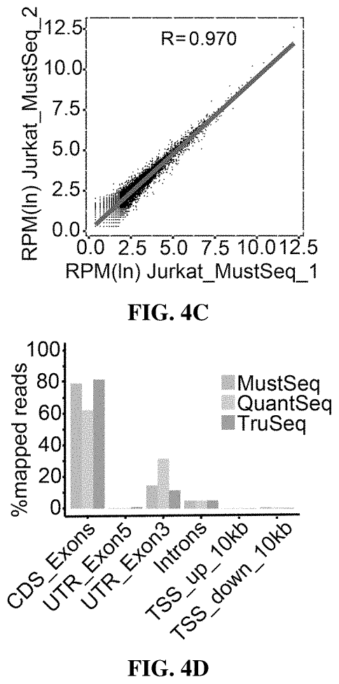

When the library is constructed by PCR amplification, an E-gel may be used to select the size of library fragments, (e.g., 400-1000 bp). Other size selection systems may also be applicable, such as the AMPure XP beads. Illumina HiSeq X10 platform may be used to carry out the 150 bp paired-end (PE150) sequencing. The outputs of the sequencing result are read 1 and read 2, where read 1 contains the information of UMIs, sample barcodes, and batch indexes while read 2 contains sequence information derived from the mRNA transcripts. The probability of the first six bases of read 1 shown on the chart validates the design of the present embodiment of UMI as NBBNBB or another longer size when applied, which may be followed by a six-bases barcode and poly-dT (as shown in FIG. 4A). In addition, the probability of four bases (A, T, C, G) in read 2 in every site is approximately 25% (as shown in FIG. 4B).

After library construction is complete, in a step 212, paired-end sequencing may be performed on the library.

Gene Detection and Read Distribution of MustSeq

MustSeq was applied to a total RNA of leukemia cell line Jurkat to check consistency and feasibility. To ensure reproducibility, duplications were performed on a same RNA pool. Using Cutadapt to remove adapter sequences and low-quality reads, 4,100,595 and 5,489,714 pairs of reads were obtained from these duplicates. After trimming and filtering the reads, Tohat2 was applied to map with the reads with the human genome GRCh38. Expression levels were then determined by calculating the number of times that the sequence of each gene was read. Normalization was performed with the number of reads per million mapped reads (RPM). As shown in the saturation curve of FIG. 19B, 0.5M of clean reads resulted in detection of over 10,000 genes, and 1.0M and 5M reads detected more than 1,2000 and 1,5000 genes respectively and close to saturation. The clean Q30 base rate was near 100%, and the sequencing quality was also satisfied (as shown in FIG. 19C and FIG. 19D). Based on the matrix obtained, their Pearson correlation coefficient R was 0.970 (as shown in FIG. 4C), indicating that the data obtained by MustSeq from the duplicate tests of the same RNA pool were highly qualified and reproducible.

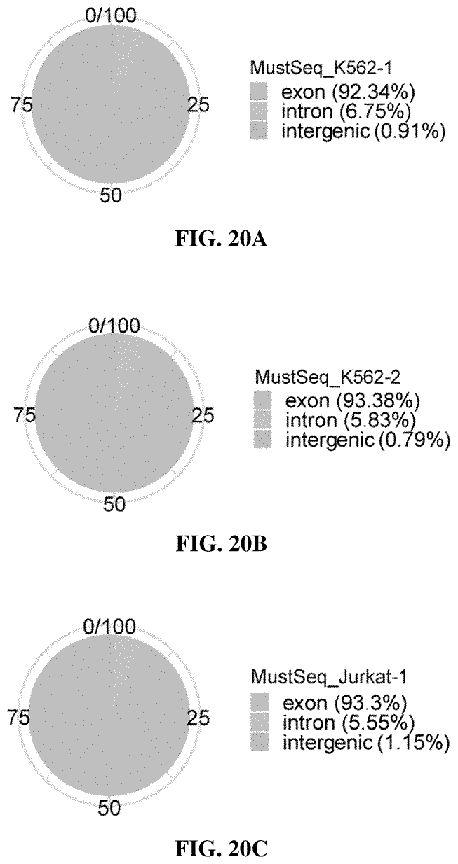

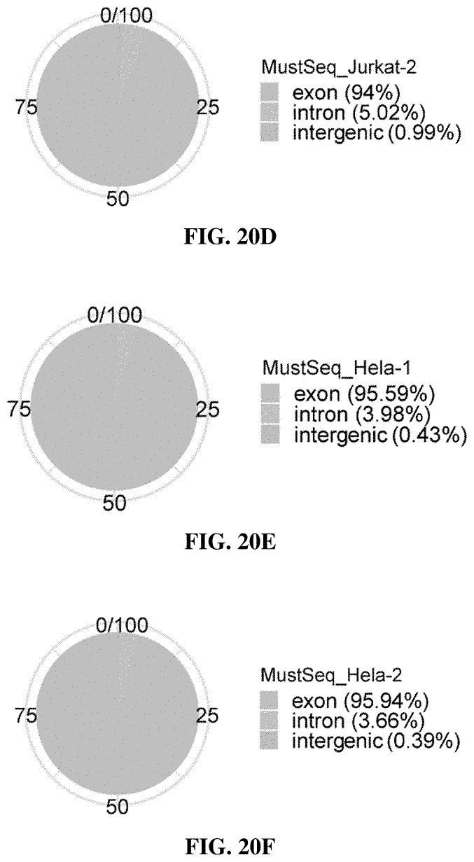

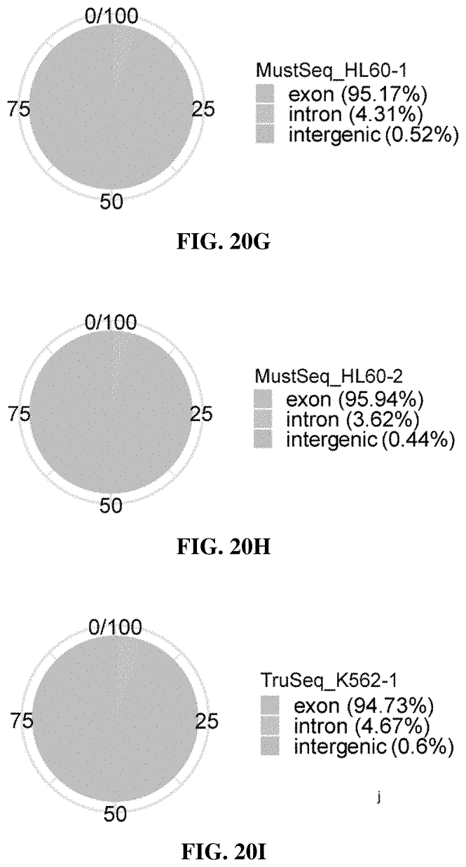

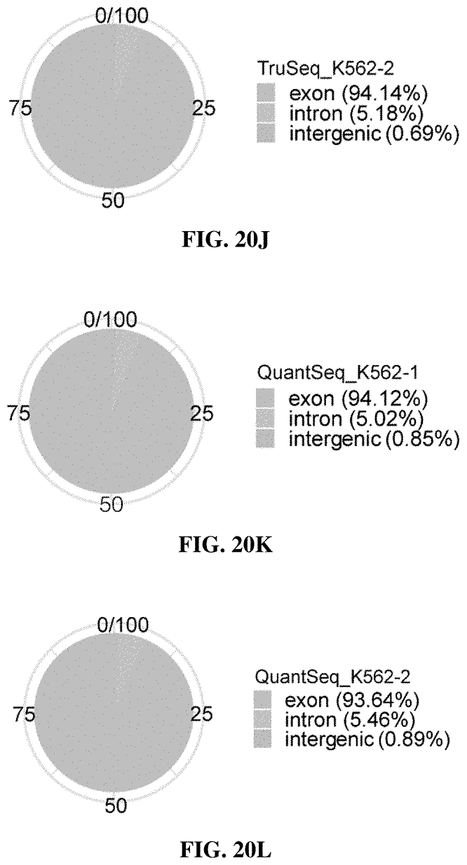

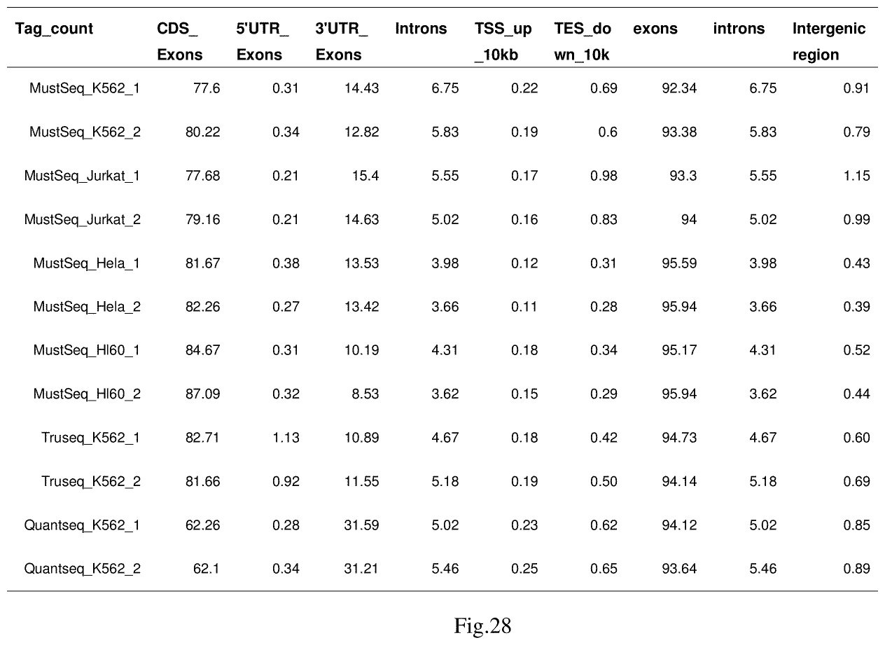

To further characterize the sequence merits of the method, gene region alignment results (in percentage form) of exon, intron, and 10 kb of transcription start sites (TSS) as well as downstream transcription termination/ending sites (TTS/TES) were counted. The results indicated that there were approximately 94% reads aligning to the exon region (as shown in FIG. 20D and FIG. 28), 79.16% to coding sequence (CDS), 14.63% to 3′ UTR (untranslated region), and little to 5′ UTR (as shown in FIG. 4D). As may be appreciated by one skilled in the art, pre-mRNA needs to be processed and spliced to remove introns, so most of the matured mRNAs are supposed to be assembled from exons. As expected, most reads were matched to the exons. The percentage of reads to introns was 5.02%, while only 0.99% mapped to intergenic regions. Due to alternative splicing or mis-priming, a few intron regions were detected. This indicates that the sequence coverage, specificity and orientation of MustSeq matched the expected results.

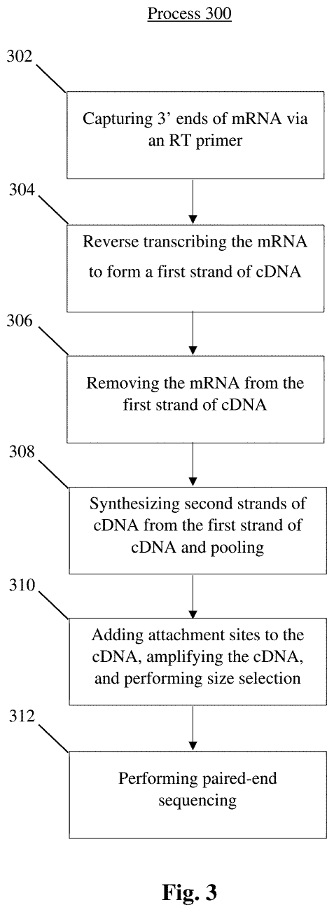

FIG. 3 illustrates a flowchart of an exemplary method for performing RNA library construction using MustSeq, in accordance with an embodiment of the present disclosure. Process 300 begins with a step 302 wherein 3′ ends of mRNA are captured using an RT primer. The RT primer may include an oligo-dT, a sample barcode, a unique molecular identifier (UMI), and a 5′ Illumina adapter. In a step 304, the mRNA may be reverse transcribed to form a first strand of cDNA. The mRNA may be removed from the first strand of cDNA in a step 306 of process 300. To remove the mRNA, RNase and heat-assisted disassociation may be used. In a step 308, second strands of cDNA may be synthesized from the first strand of cDNA, and the cDNA (including the first strand and second strands) may be pooled. The second strand synthesis may be performed using a chimeric primer and Klenow Fragment lacking exonuclease activity. The second strands of cDNA may include double stranded cDNA and single stranded cDNA. In a step 310, attachment sites may be added to the cDNA, the cDNA may be amplified, and size selection may be performed on the amplified cDNA. A P5 primer may be used to add a P5 attachment site at a 5′ Illumina adapter and a P7 primer may be used to add the P7 attachment site and the primer at the 3′ adapter. The cDNA may be amplified using PCR, and an E-gel may be used to perform size selection on the amplified cDNA to select cDNA fragments with 400-1000 base pairs to be included in the sequencing library. In a step 312, paired-end sequencing may be performed on the sequencing library.

Comparison of MustSeq with QuantSeq and TruSeq

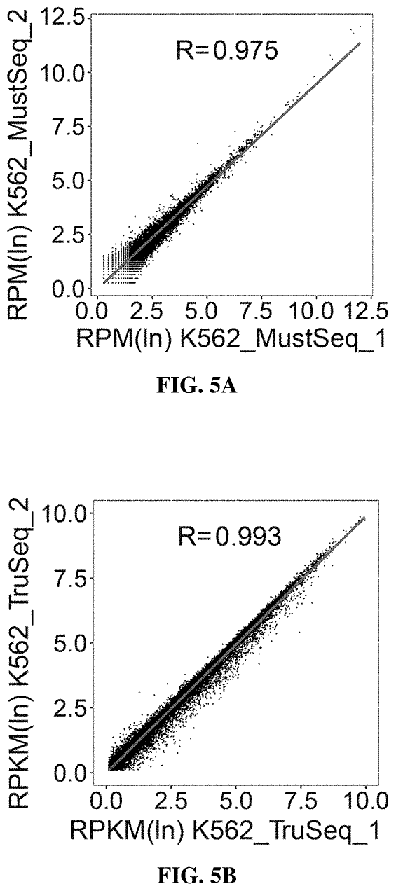

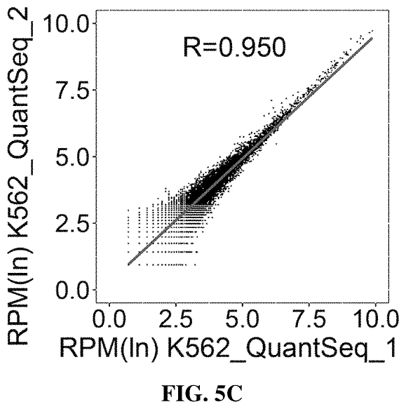

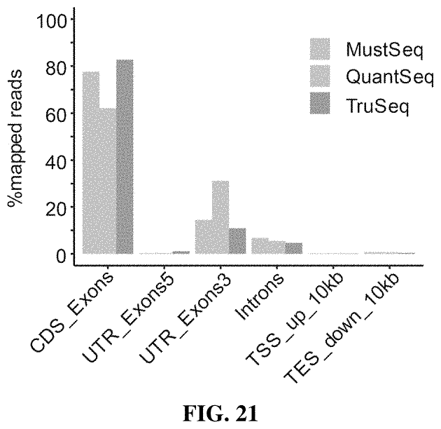

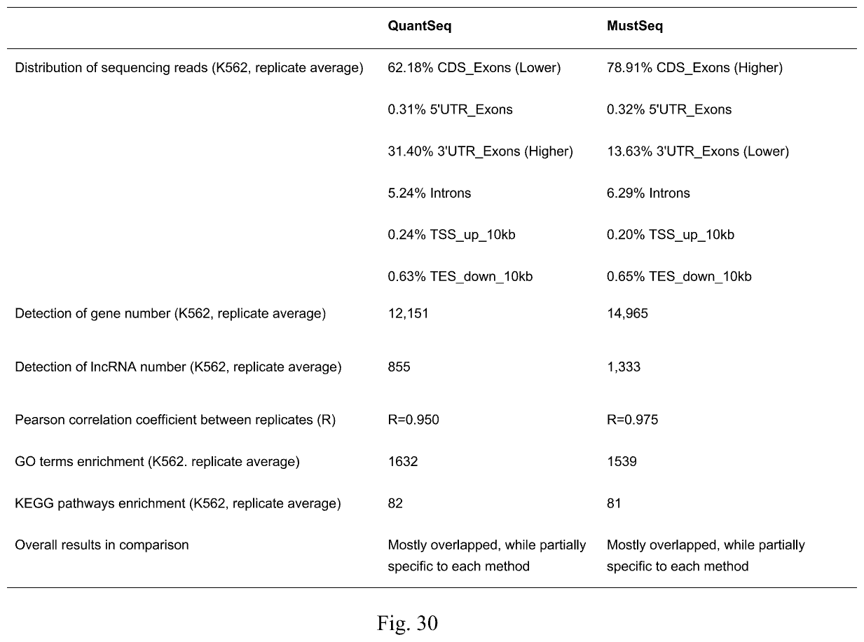

Having demonstrated that MustSeq detected strand and orientation specifically, ability to quantify transcription was subsequently tested. As described herein, MustSeq was applied to cell line K562 and compared the procedure and the results with that from QuantSeq (as shown in FIGS. 29 and 30), and also from TruSeq (downloaded from the Encyclopedia of DNA Elements, ENCODE, database). As expected, MustSeq was highly reproducible between different tests (with a Pearson correlation coefficient R=0.975, as shown in FIG. 5A) comparable to both TruSeq (R=0.993, as shown in FIG. 5B) and QuantSeq (R=0.950, as shown in FIG. 5C). Meanwhile, the three methods showed similar distribution of reads, including exon, intron and intergenic regions, and all with less than 1% mapping to a 5′ UTR exon (as shown in FIGS. 20A-20B, FIGS. 20I-L and FIG. 21). On the other hand, approximately 80% and 12% reads of both MustSeq and TruSeq were mapped to CDS exon and 3′UTR exon, respectively. In contrast, 60% and 30% reads of QuantSeq were mapped to CDS exon and 3′ UTR exon respectively (as shown in FIG. 28).

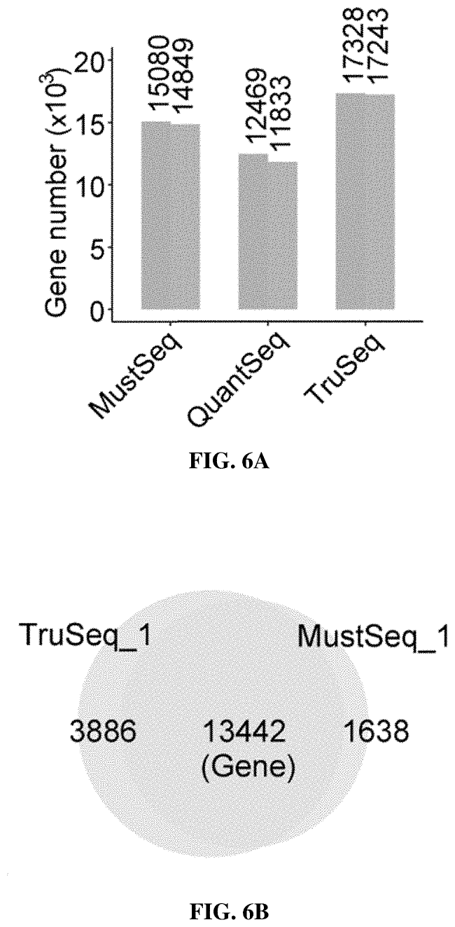

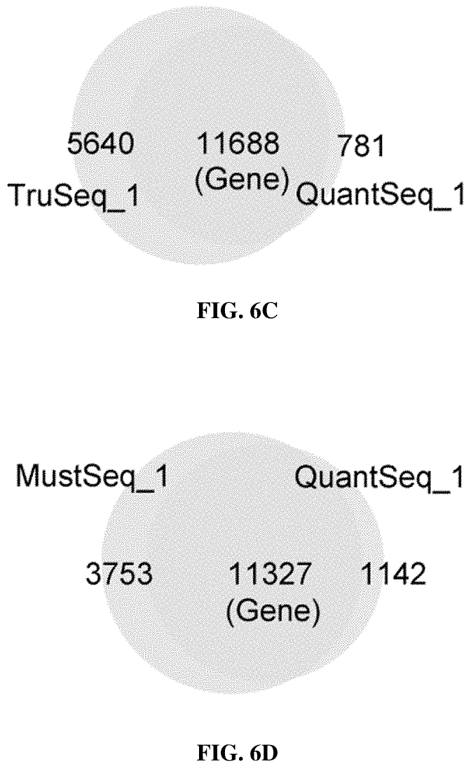

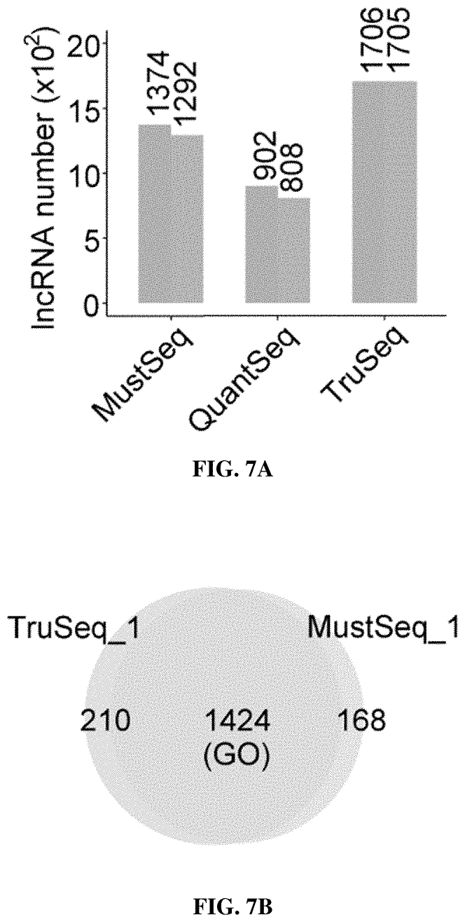

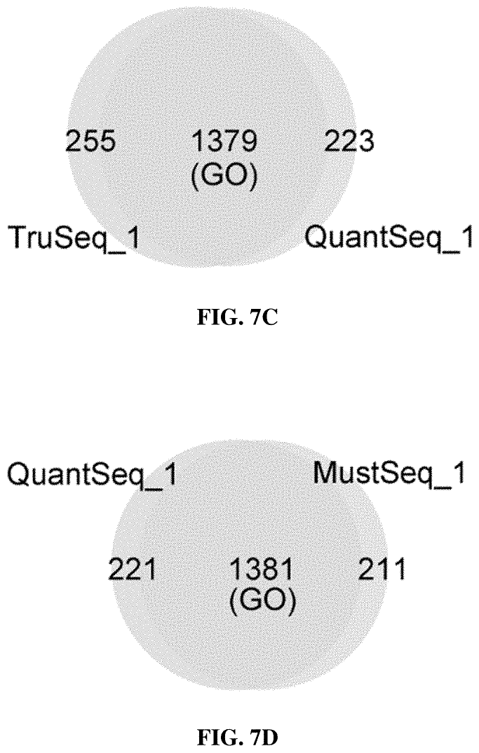

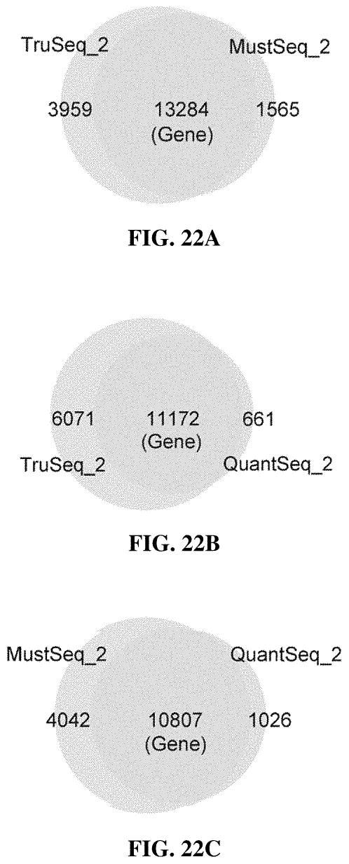

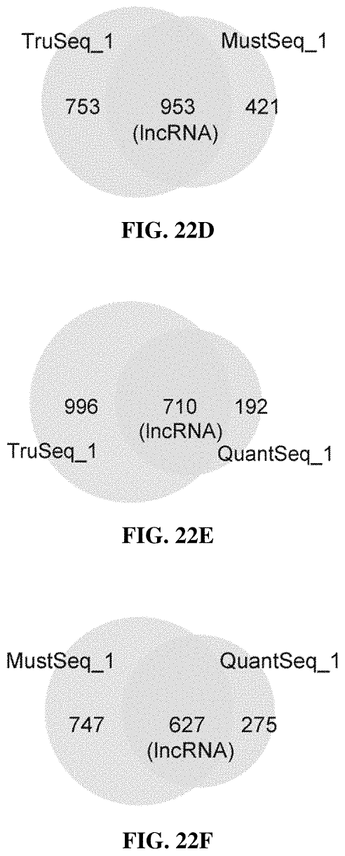

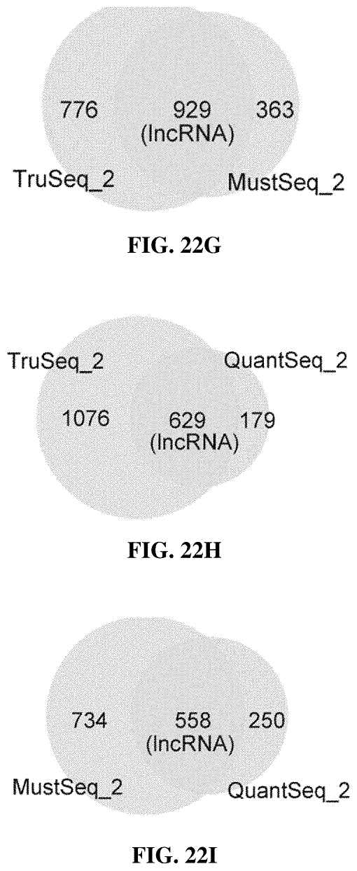

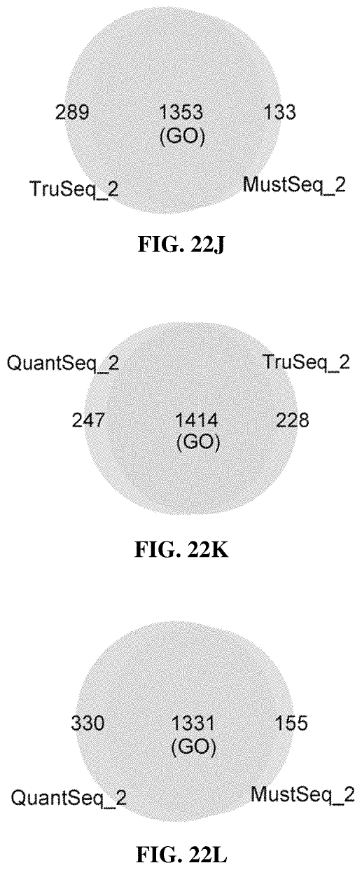

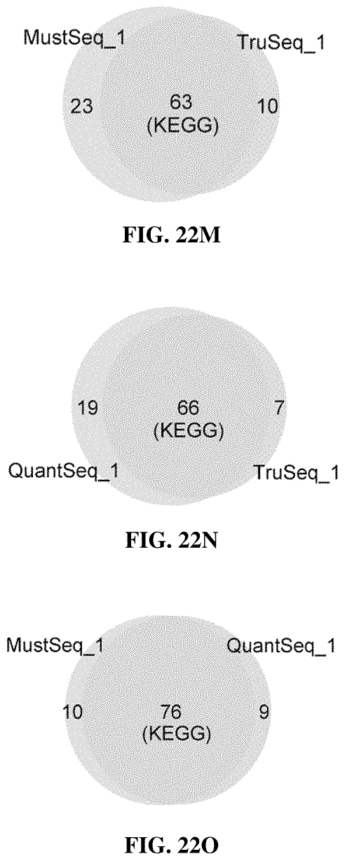

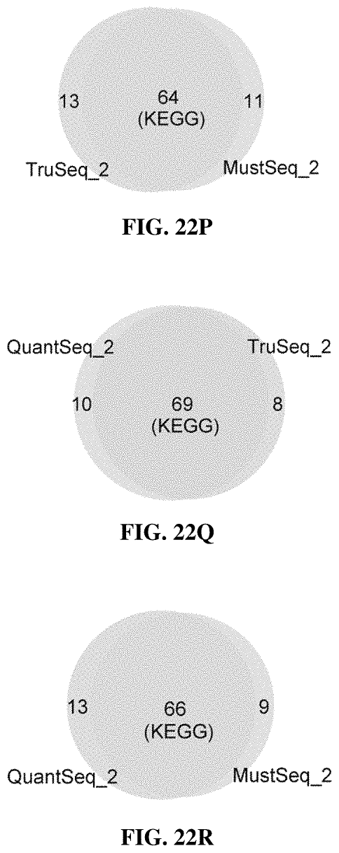

On average for the duplicate tests of K562, 14,965 mRNA genes were identified (RPM>0) within the duplicates of K562 by MustSeq, 12,151 genes were identified by QuantSeq (RPM>0), and 17,286 genes were identified by TruSeq (RPKM>0.1, as shown in FIG. 6A). It was found that 1,602 genes were specifically detected by MustSeq and 3,923 genes by TruSeq, while 13,363 genes were commonly detected by both methods (as shown in FIG. 6B and FIG. 22A). Almost all, with a few exceptions, of the genes specifically detected in MustSeq (no expression was detected in TruSeq) are at a very low level in MustSeq, and vice versa. Compared the detected number of genes between TruSeq and QuantSeq, beyond the 11,430 genes detected by both, TruSeq specifically detected an additional 5,856 genes, while QuantSeq detected 721 specific genes average in replicates (as shown in FIG. 6C and FIG. 22B). A similar comparison between MustSeq and QuantSeq showed that the former method detected an additional 3,898 genes other than the commonly detected 11,067 genes, while the latter only detected 1,084 specific genes (as shown in FIG. 6D and FIG. 22C). With both 3′ end or full-length RNA-seq, the methods all detected long non-coding RNAs (lncRNAs). The average number of lncRNAs detected by MustSeq, QuantSeq and TruSeq in the two replicates of cell line K562 was 1,333, 855, 1,706, respectively (as shown in FIG. 7A). MustSeq and TruSeq each detected about 45% of lncRNAs in common when these two methods were compared, and MustSeq detected additional 19% specific lncRNAs (TruSeq detected 36% additional lncRNAs) (as shown in FIG. 22D and FIG. 22G). However, when TruSeq was compared with QuantSeq, it was found that 44.98% more lncRNAs were detected in TruSeq than in QuantSeq, beyond those detected in common (as shown in FIG. 22E and FIG. 22H). MustSeq also detected 30% more lncRNAs than QuantSeq (as shown in FIG. 22F and FIG. 22i).

Gene Ontology (GO) enrichment analysis was performed using R package Cluster Profiler for genes. Firstly, TruSeq and MustSeq were enriched to 1,638 and 1,539 GO terms respectively, of which 1,389 terms were found to be commonly enriched by both methods (as shown in FIG. 7B and FIG. 22J). TruSeq enriched only 99 specific terms more than that of MustSeq. These results show that although MustSeq detects only the 3′ end of mRNA, its enriched GO terms overlap with the conventional full-length method by 77.63%. Secondly, on average QuantSeq detected 1,632 terms. Besides the 1,397 terms detected in common (73.38%), TruSeq specifically detected 242 unique terms, while QuantSeq detected 235 unique terms (as shown in FIG. 7C and FIG. 22K). Thirdly, the comparison between MustSeq and QuantSeq showed that the former method detected 188 terms uniquely, while the latter method detected 271 specific terms (as shown in FIG. 7D and FIG. 22L).

The Kyoto Encyclopedia of Genes and Genomes (KEGG) pathways were similar to GO in the K562 analysis, although the KEGG pathways were less than GO terms in number scale (as shown in FIGS. 7B-7D and FIGS. 22J-R). When KEGG pathways were compared among the tested methods, TruSeq detected only 12.50% of specific pathways while MustSeq detected 18.48% of specific pathways, and the overlap rate between two methods was 69.02% (as shown in FIG. 22M and FIG. 22P). QuantSeq specifically enriched 7.82% more KEGG pathways than TruSeq (as shown in FIG. 22N and FIG. 22Q); the overlap of KEGG pathways between MustSeq and QuantSeq was as high as 77.60% (as shown in FIG. 22O and FIG. 22R).

Validation of MustSeq on Transcriptomic Analysis for Cancer Cell Lines

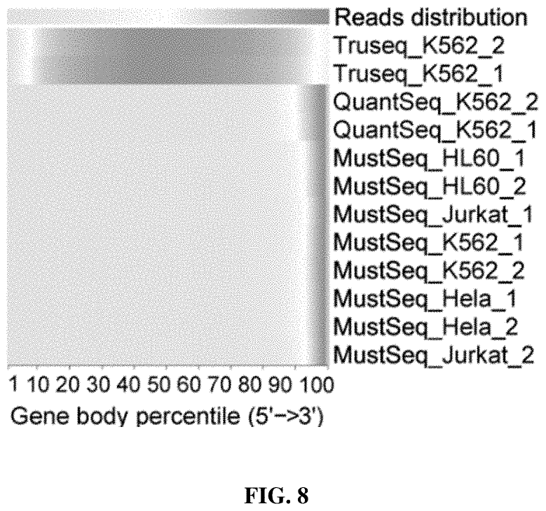

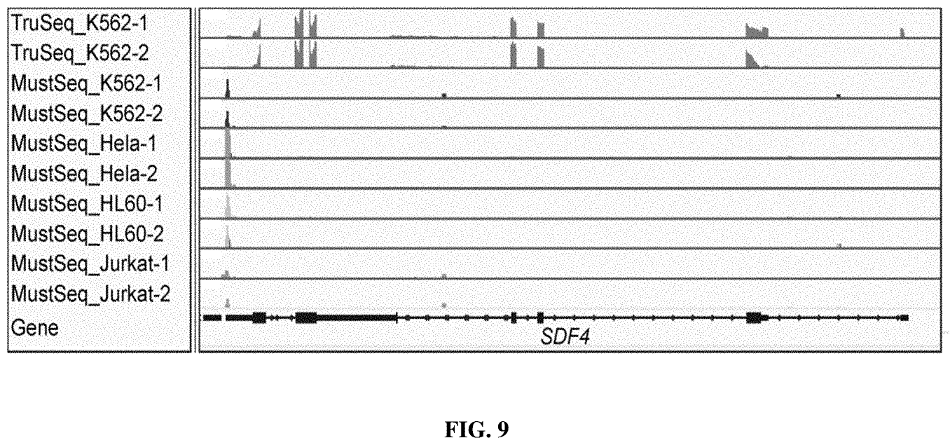

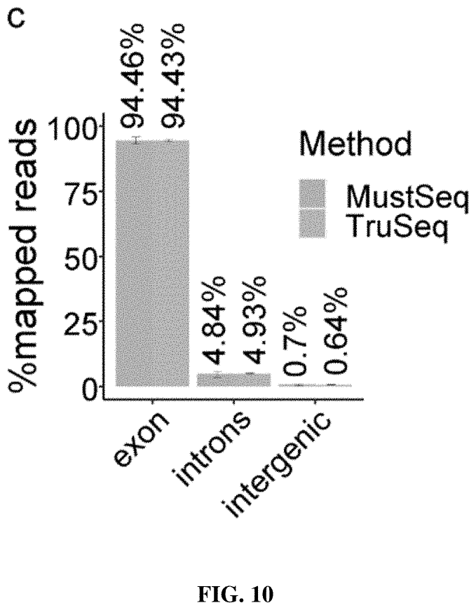

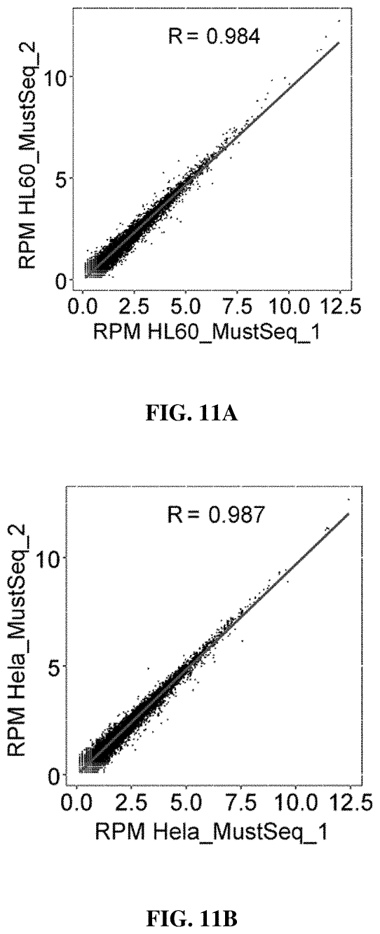

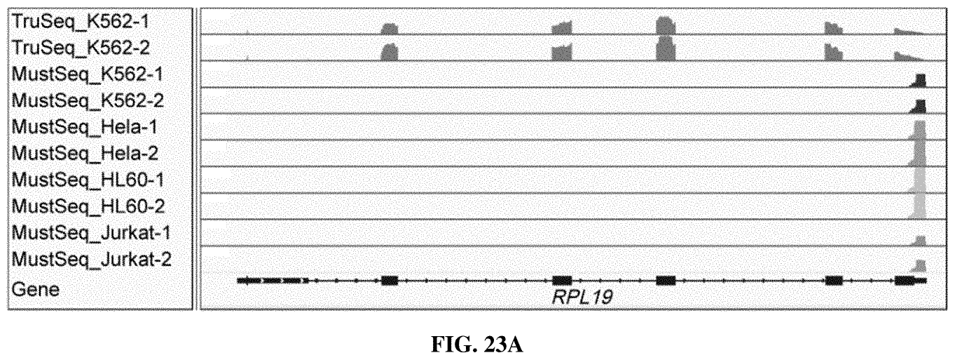

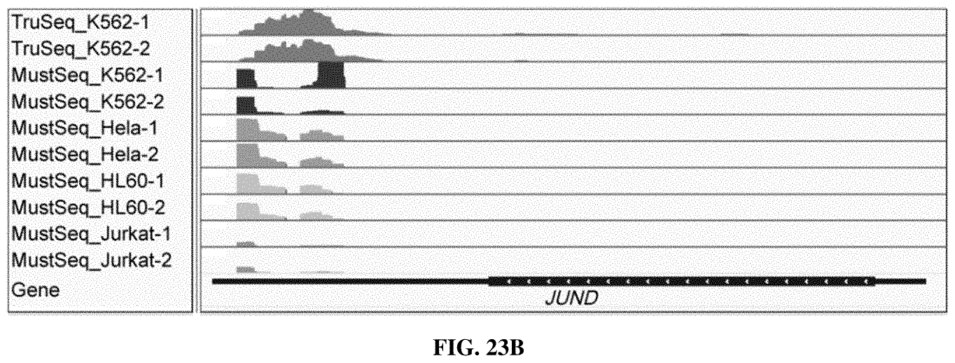

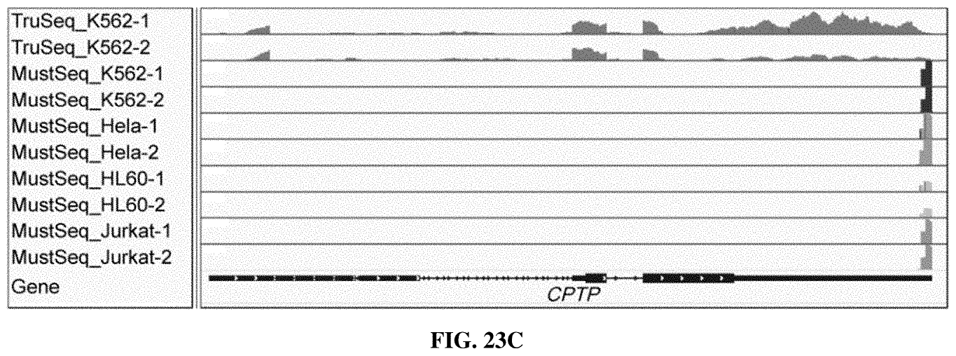

After successfully testing the MustSeq method on Jurkat and K562 cells, MustSeq was further applied on 4 cancer cell lines for further validation, including K562, Jurkat, HL60 and Hela. Firstly, MustSeq was used to measure duplicates per cell line. The sequencing reads were all perfectly matched to the 3′ end of the genes, indicating that MustSeq accurately captured the 3′ end of the mRNA (as shown in FIG. 8). Integrative Genomics Viewer (IGV) was used to show that the reads of the TruSeq method covered the full length of the transcript of a gene, SDF4, while MustSeq specifically covered the 3′ end coding region of SDF4 (as shown in FIG. 9), and similar results were found for genes RPL19, JUND and CPTP (as shown in FIGS. 23A-C). Secondly, the percentage of reads mapping to exon, intron and intergenic regions were similar across each method, indicating a high consistency (as shown in FIGS. 20A-H). It is worth noting that the distribution of MustSeq reads to detect exons (CDS, 3UTR, 5UTR), introns, TSS and TES of genes was close to that of TruSeq (as shown in FIG. 10), while QuantSeq showed a relative different distribution (as shown in FIG. 4D, FIG. 21, and FIG. 28). Fourthly, when comparing the gene expression levels of duplicates from the same cell line, the average Pearson correlation coefficient of the four cell lines was 0.979, indicating that the MustSeq data is highly reproducible (as shown in FIG. 4C, FIG. 5A, and FIGS. 11A-B).

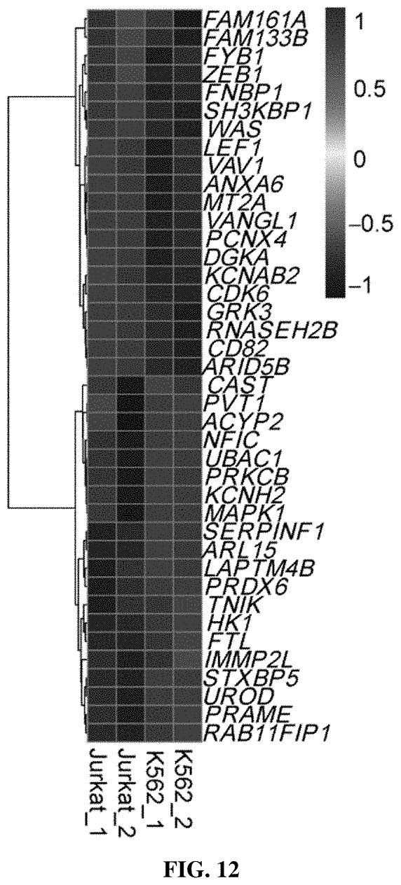

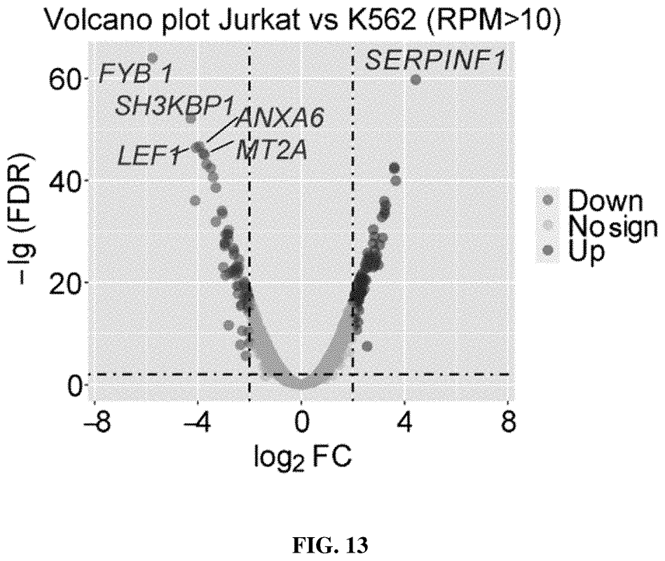

MustSeq may also identify biological DEG between cell lines. By examining their transcriptomes, it was found that 40 genes with a 2-fold change of expression between lymphoblastic leukemia cell line Jurkat and myeloid leukemia cell line K562 (p<0.01, FDR-corrected). The results showed that the expression level of FYB1, CD82, ARID5B, ZEB1 and other genes were relatively higher in Jurkat than K562, which was consistent with early reports that the expression levels of CDK6 [33], LEF1 [34] and ZEB1 [35] were significantly upregulated in acute lymphoblastic leukemia cell lines or patient samples. Researches also showed that the expressions of genes PRAME [36], UROD [37] and PVT1 [38] in myeloid leukemia or myeloid cell lines were significantly up-regulated, which also supported the results obtained in MustSeq (as shown in FIG. 12). With volcano plot, a set of genes was detected with the most significant difference in expression between the 2 cell lines (|log₂ FC|>2, FDR<0.01; as shown in FIG. 13). The MustSeq method yielded sufficient, and reliable DEGs, which indicated that it is suitable for studying DEG between different conditions and identifying up-regulated or down-regulated genes.

Dissection of Transcriptomic Profile of Hepatocarcinoma in a Mouse Model Using MustSeq

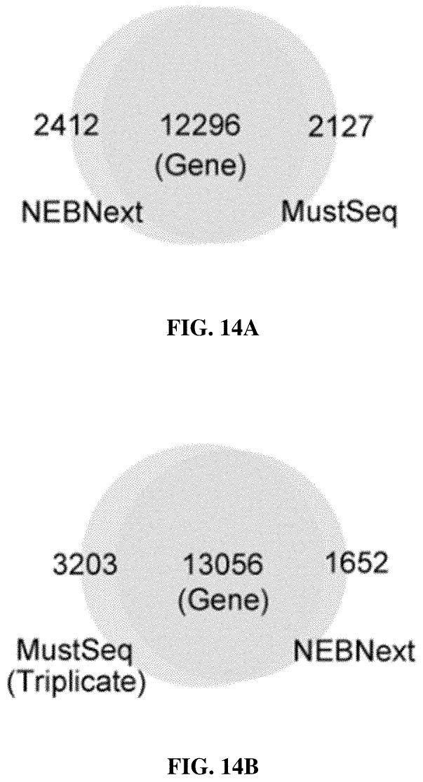

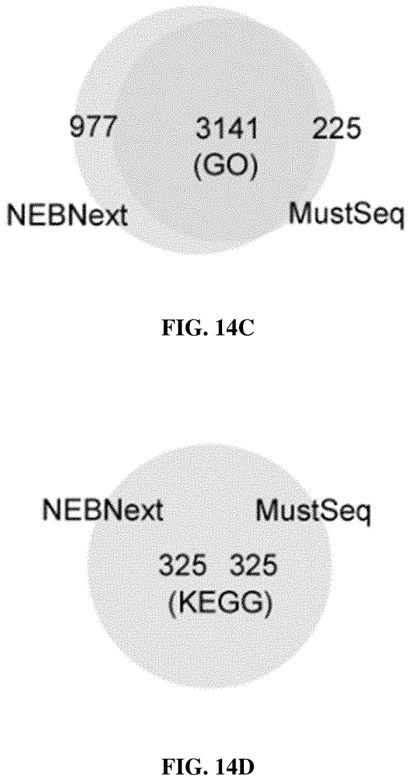

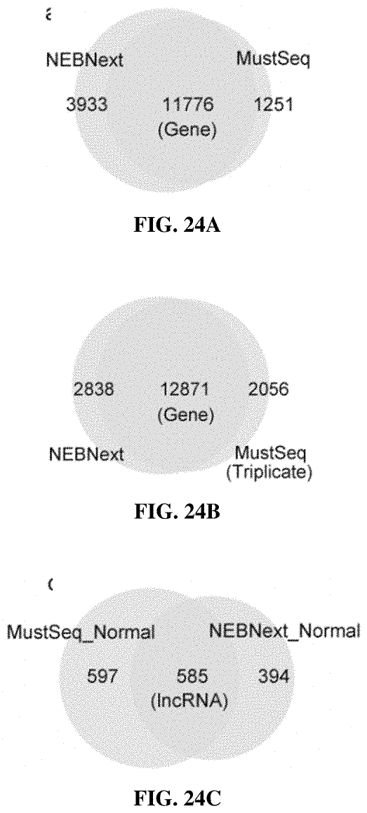

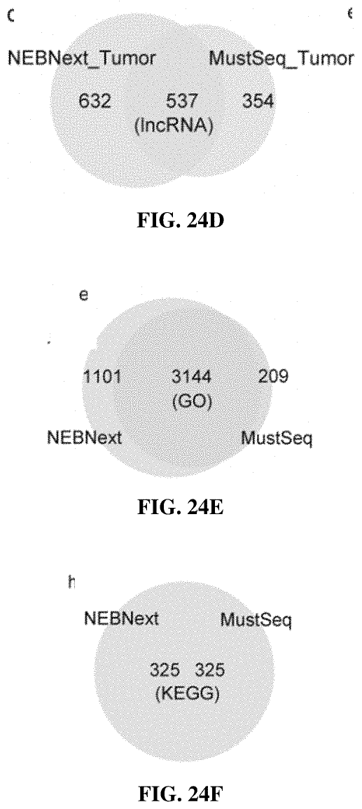

MustSeq was also applied to the livers of a mouse model. B6-Hippo11^{e(CAG-LNL-c-MYC)|Smoc}, which was HCC generated by conditionally overexpressed c-Myc in the liver, versus a healthy liver control, was used to test whether the method of the present embodiment accurately detects expressions of oncogenes in native tissue. NEBNext was applied to these samples in parallel for comparison. Similar to the TruSeq method, NEBNext detects more genes than MustSeq in both HCC and healthy liver, but more than 11,700 genes were commonly detected by both methods (as shown in FIG. 14A and FIG. 24A). In a healthy liver, the number of genes specifically detected by MustSeq alone was 2,127 and by NEBNext alone it was 2,412 genes. In addition, for HCC, the number of genes specifically detected by MustSeq was 1,251, and by NEBNext it was 3,933 genes. The collection of genes of triplicates was compared with MustSeq to a test with NEBNext. The number of genes specific detected by MustSeq with a collection of triplicates was 1,551 more than that by the NEBNext with a single test for healthy liver (as shown in FIG. 14B), while it was only 782 more genes detected by NEBNext with single test than MustSeq for HCC with triplicate test (as shown in FIG. 24B). In this case, the genes commonly detected by both methods was 13,056 and 12,871 genes in hepatocarcinoma and healthy liver, respectively. In addition, NEBNext detected, on average, only about 2% more lncRNAs than Mustseq (as shown in FIGS. 24C-D). Here especially, the high efficiency in cost and labor of MustSeq is advantageous over conventional means.

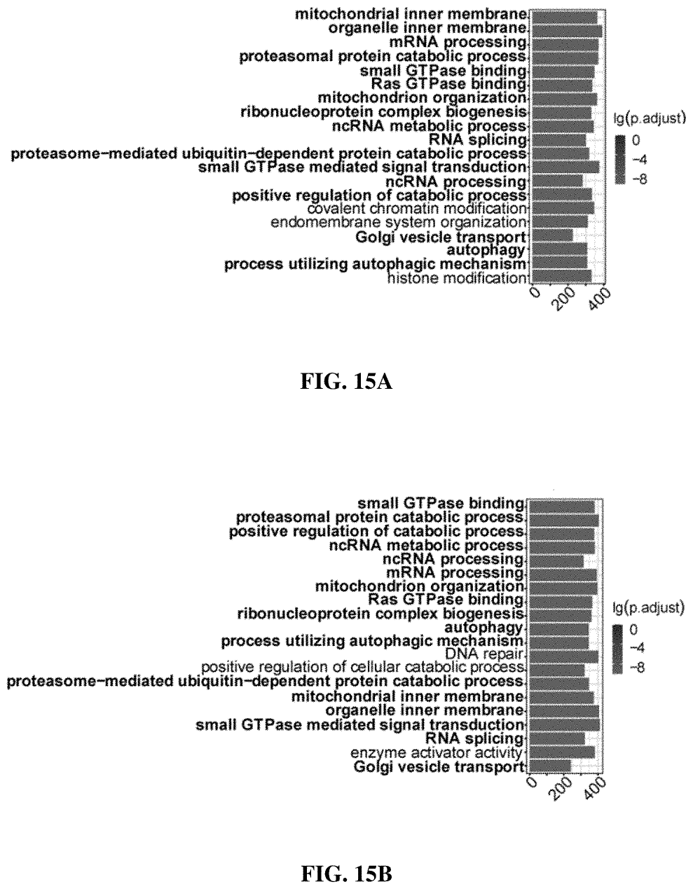

When GO term enrichment was carried out using data from healthy liver samples, NEBNext detected 4,118 terms and MustSeq detected 3,366 terms, with 752 more terms detected using NEBNext than when using MustSeq. Both methods detected 3,414 terms in common, while NEBNext specifically detected 977 terms, MustSeq specifically detected 225 terms (as shown in FIG. 14C). The 17 terms out of the top 20 most significant terms were in common between the two methods, including mRNA processing, ribonucleoprotein complex biogenesis, proteasomal protein catabolic process and so on (as shown in FIGS. 15A-B). When using KEGG database for pathway enrichment, it was found that the pathways detected by MustSeq and NEBNext were exactly the same, with 325 pathways (as shown in FIG. 14D).

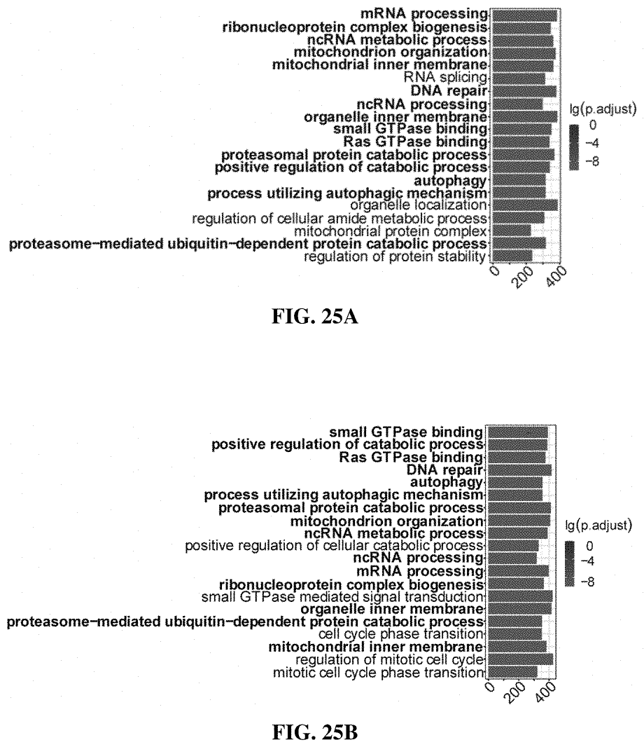

Next, the present embodiment enriched GO terms with the data of HCC and healthy livers. NEBNext detected 4,245 terms and MustSeq detected 3,353 terms, both with single test. NEBNext detected 686 more terms than MustSeq. Both methods detected 3,144 terms in common, while there were 1,101 terms specifically detected by NEBNext, and 209 terms by MustSeq (as shown in FIG. 24E). Among the top 20 most significant terms, 15 were same for the two methods, including DNA repair, positive regulation of catabolic process and mRNA processing (as shown in FIGS. 25A-B). The reliability of MustSeq was again confirmed that MustSeq and NEBNext detected exactly the same number of and type of 325 pathways in HCC and healthy liver (as shown in FIG. 14D and FIG. 24F).

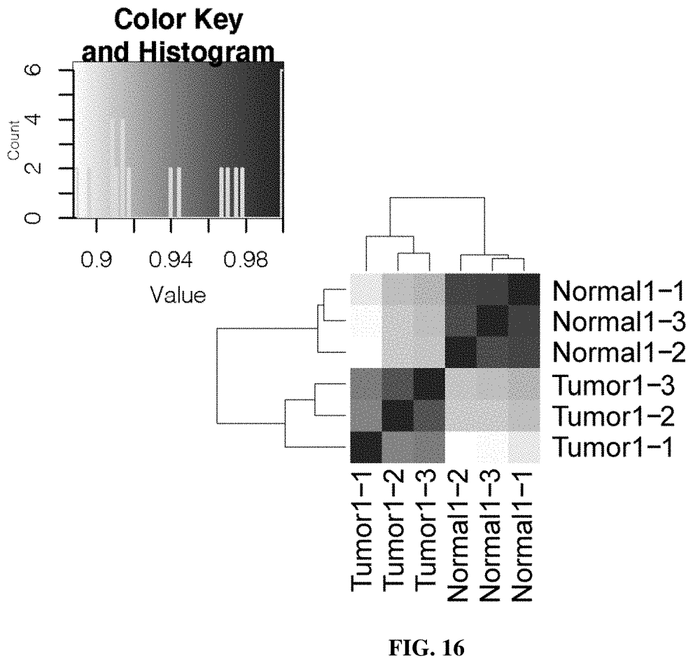

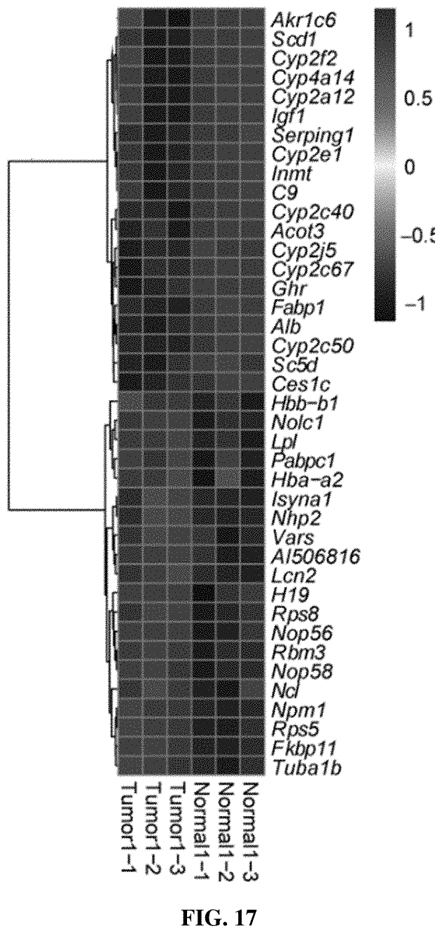

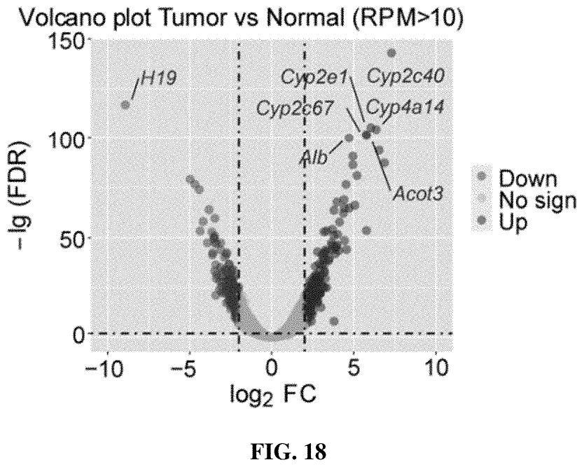

To examine the reproducibility of MustSeq in biological replicates and the DEGs between conditions, the present embodiment analyzed gene expression levels (RPM>0), and calculated Pearson correlation coefficients among all samples. The results showed, in the sequencing data of HCC and healthy liver (each with triplicates), that the Pearson correlation between duplicates was much higher, and the Pearson correlation between HCC and the control was lower (as shown in FIG. 16). Next, the present embodiment performed DEG analysis on the two groups and took the top 20 genes with the most significant differences to make a heat map (p-value <0.01, |log₂ FC|>2, RPM>10; as shown in FIG. 17). As shown, the top 20 DEGs, including Pabpc1 [39], Fkbp11 [40], H19 [41], etc., were significantly up-regulated in HCCs; while the bottom 20 DEGs, including Ghr [42], Cyp2c67 [43], Scd1 [44], etc., were greatly down-regulated in HCCs. These results are consistent with early reports, proving that the differential gene map obtained by the MustSeq library construction method are highly reliable. From the volcano map (as shown in FIG. 18), it was found that H19 has the smallest FPR (false positive rate), and its differential expression between carcinoma and healthy liver was supported in Huang et al [41].

Discussion

Transcriptomic profile is the molecular basis regulating human health and diseases [2]. However, current technologies have yet to show variants of deficiencies when sample number is large, particularly labor-intensive, time-consuming, and high-cost methods for both library construction and sequencing. In response, MustSeq is developed as an independently designed library preparing method for RNA-Seq. MustSeq was tested with four cancer cell lines and sets of liver tissues from a mouse model. The results show that MustSeq is successful in design, and efficient and reliable in practice.

MustSeq Brings High Efficiency to RNA-Seq.

MustSeq provides an efficient choice for studying transcriptome when DEGs are the major expectation. The key advantage of MustSeq over other full-length mRNA-seq methods, such as TruSeq [13-17] and NEBNext [18, 19], 3′ end bulk method [21,45], and 3′ end single-cell sequencing library construction method [30], is that conventional full-length mRNA-seq methods do not include steps for adding a tag sequence on the 5′ end of the mRNAs or their corresponding cDNA. MustSeq may be used to directly synthesize multiple second cDNA strands for each first strand through a primer set including a 3′ random priming end and 5′ anchoring tag. The anchoring tag, when combined with the poly-dT primers possessing another anchoring PCR tag plus a sample barcode on the other end of the cDNA, allows PCR amplification with two primers in the following step to complete the library construction. As such, the entire library construction processes of multiple samples may be performed in parallel, only requiring one round of PCR amplification immediately following first strand and second strand cDNA synthesis, making MustSeq highly convenient and efficient. The whole process of MustSeq takes approximately 3.5 hours. In contrast, the conventional methods need seven experimental frameworks and multiple reaction steps to prepare the library. For example, the TruSeq method includes a large number of steps, and may take 11 hours to complete. More importantly, the conventional methods for bulk RNA-seq treat each sample independently from the start of library construction to the completion of library construction. In contrast, MustSeq, immediately after first strand synthesis, may be used to pool multiple samples each with a unique sample barcode to one tube for all downstream processes, and decode each sample after sequencing is completed, which significantly improve operation efficiency as well as cost efficiency, especially when the sample number is large. Furthermore, theoretically sequencing the 3′ ends of mRNAs requires detection of roughly 100-150 bp or shorter as long as the read is mappable uniquely to a gene, while full-length mRNA sequencing with other conventional methods needs to cover more than 2000-3000 bp in average, hence the sequencing cost could be decreased by approximately twenty fold.

MustSeq Accurately Quantities the Transcriptomic Profile.

Due to the focus on 3′ end sequencing of transcriptome, MustSeq may be used for identification of DEGs over the whole mRNA transcriptome, and additionally detection of alternative transcripts controlled by the 3′ end sequences. In contrast to other bulk RNA-Seq methods available, this greatly simplifies the estimation of expression difference because no normalization by gene length is necessary [22]. Indeed, single cell RNA-seq using a 3′ end sequencing strategy on single cells on 10× Genomics Chromium may be frequently applied.

Even though MustSeq detects only the 3′ end of mRNAs, the capability of MustSeq at detecting the number of genes and lncRNAs, enriching GO terms and KEGG pathways, is just slightly lower and very close to TruSeq method and NEBNext, two conventional methods for full length RNA-seq. Meanwhile, the 3′ end methods capture additional transcripts that are missed with the full-length RNA-sequencing methods, which may be attributed to the high 3′ end enrichment by MustSeq. An early comparison revealed that a different 3′ RNA sequencing method is faithful while it detected more short transcripts than full-length RNA sequencing [46]. Therefore, MustSeq well reflects the identification of DEGs between distinct samples, in spite of capturing approximately 87% of genes detected by a TruSeq, while MustSeq also detected an additional in average 8% genes missed in TruSeq in 2 representative tests (as shown in FIGS. 6B and 22A). The drawback of fewer genes detected by MustSeq could be compensated by a collection of replicates, taking advantage of the high efficiency in cost and labor of MustSeq. For example, a triplicate collection of MustSeq consistently detects more genes than a single test of NEBNext (as shown in FIG. 14B). Meanwhile, with GO analysis by the R-package clusterProfiler, 77.63% of enriched GO terms are similar to TruSeq, while MustSeq and TruSeq each detected roughly 8-14% additional specific GO terms (as shown in FIG. 7B and FIG. 22J). Nevertheless, it should be noted that MustSeq enriches the same KEGG pathways perfectly consistent to NEBNext in both HCC and healthy livers (as shown in FIGS. 15B and 24F).

In practical analysis, differential genes detected by MustSeq in the c-Myc initiated HCC are consistent with the study by Lu et al. [47], which states that the expression of Tuba1b is up-regulated in liver cancer tissues and proliferating liver cancer cells. The results of the present disclosure are also supported by the study [40] that the expression level of Fkbp11 in liver tumor tissue is higher than that in the control tissue (as shown in FIG. 17). Taken together, the DEG results based on 3′ end transcriptome generated from MustSeq are comparable to the full-length transcriptome data of conventional methods.

MustSeq demonstrates unique features comparing to Tre-seq, NEBNext and QuantSeq.

Compared with TruSeq and NEBNext RNA-seq, MustSeq (just like QuantSeq and other terminally representative RNA-seq methods) detects fewer genes and GO terms when no compensation effort was applied. It is worth mentioning that their comparisons may be bade between a full-length method and a 3′ end method [48]. The difference in number of genes detected by the 3′ end sequencing method CEL-Seq2 [30] and the full-length sequencing method Smart-Seq2 [49] is similar to the results found in the present embodiment. The average number of genes detected by single-cell CEL-Seq2 was 7,536, while the average number of genes detected by single-cell Smart-Seq2 was 9,138. After combining the data of 65 single-cells from these two methods, the total number of genes detected by CEL-Seq2 was 19,000, and the total number of genes detected by Smart-Seq2 was 21,000. This indicates that the number of genes detected by the 3′ end sequencing method is generally lower than that with the full-length sequencing methods. It is known that RNA isoform variability is resulted in alternative choice of transcription start sites (TSSs) [50], transcription end sites (TESs) [51], exons, and sometimes intron inclusion. Therefore, the main cause for the less genes detected with 3′ end RNA-seq is that intrinsically 3′ end RNA-seq methods only capture the splicing forms solely associated with the 3′ terminal sequences, while full-length sequencing captures many kinds of splicing forms. While MustSeq may intrinsically detect fewer genes when compared to full-length sequencing methods, MustSeq may be more efficient than conventional full-length sequencing. Full-length RNA-seq also enables single nucleotide mutation detection and allele expression of the transcriptome, which is a useful message for study of genetic and somatic disease. On the other hand, this may be an advantage for analysis of transcriptomic terminals that it enabled 3′ UTR to be highly detected, in addition the strand specificity of the transcripts was determined. With further optimization to improve sensitivity, MustSeq may be a medium- or high-throughput, bench-top approach for partially degraded RNA (such as formalin-fixed paraffin-embedded tissue) and bench-top single-cell RNA-seq. MustSeq is robust, financially efficient, and may be adapted to be widely applicable in most molecular biology laboratories or clinical laboratories.

MustSeq, similar to QuantSeq, both of which are methods for 3′ end RNA-seq, detects slightly less number of genes, lncRNAs and GO terms than TruSeq and/or NEBNext, two conventional methods for full length RNA-seq. However, MustSeq libraries possess a relatively higher detection of genes and lncRNAs than QuantSeq. In addition, MustSeq enriches the same KEGG pathways as that detected by NEBNext in two sets of mouse liver tissues studied. Importantly, triplicate application of MustSeq may increase the detection of genes to the level of full-length RNA-seq, and may be accompanied by the advantage of reduced costs and increased efficiency. Overall, the performance of MustSeq is more similar to QuantSeq than TruSeq in regard to enrichment of GO terms and detection of number of genes and lncRNAs as well as the reads distribution along the genome, while each method has a certain preference.

### REFERENCE LIST

[1] Wang Z, Gerstein M, Snyder M. RNA-Seq: a revolutionary tool for transcriptomics. Nat Rev Genet 2009; 10:57-63.
[2] van Galen P, Hovestadt V, Wadsworth I M H, Hughes T K, Griffin G K, Battaglia S, et al. Single-Cell RNA-Seq Reveals AML Hierarchies Relevant to Disease Progression and Immunity. Cell 2019; 176:1265-81.e24.
[3] Popescu D M, Bating R A, Stephenson E, Green K, Webb S, Jardine L, et al. Decoding human fetal liver haematopoiesis. Nature 2019; 574:365-71.
[4] Zhang J, Spath S S, Marjani S L, Zhang W, Pan X. Characterization of cancer genomic heterogeneity by next-generation sequencing advances precision medicine in cancer treatment. Precis Clin Med 2018; 1:29-48.
[5] Huang R, Huang Y, Zeng G, Li M, Jin Y. Ursodeoxycholic acid inhibits intimal hyperplasia, vascular smooth muscle cell excessive proliferation, migration via blocking miR-21/PTEN/AKT/mTOR signaling pathway. Cell Cycle 2020; 19:918-32.
[6] Lupinek C, Wollmann E, Valenta R. Monitoring Allergen Immunotherapy Effects by Microarray. Curr Treat Options Allergy 2016; 3:189-203.
[7] Zhang Y, Wu X, Zhang C, Wang J, Fei G, Di X, et al. Dissecting expression profiles of gastric precancerous lesions and early gastric cancer to explore crucial molecules in intestinal-type gastric cancer tumorigenesis. J Pathol 2020; 251:135-46.
[8] Beaulieu Y B, Kleinman C L, Landry-Voyer A M, Majewski J, Bachand F. Polyadenylation-dependent control of long noncoding RNA expression by the poly(A)-binding protein nuclear 1. PLoS Genet 2012; 8:e1003078.
[9] Sun Y, Zhang Q, Liu B, Lin K, Zhang Z, Pang E. CuAS: a database of annotated transcripts generated by alternative splicing in cucumbers. BMC Plant Biol 2020; 20:119.
[10] Manipur I, Granata I, Guarracino M R. Exploiting single-cell RNA sequencing data to link alternative splicing and cancer heterogeneity: A computational approach. Int J Biochem Cell Biol 2019; 108:51-60.
[11] Piskol R, Ramaswami G, Li J B. Reliable identification of genomic variants from RNA-seq data. Am J Hum Genet 2013; 93:641-51.
[12] Dirks R A M, van Mierlo G, Kerstens H H D, Bernardo A S, Kobolak J, Bock I, et al. Allele-specific RNA-seq expression profiling of imprinted genes in mouse isogenic pluripotent states. Epigenetics Chromatin 2019; 12:14.
[13] Marques S, van Bruggen D, Vanichkina D P, Floriddia E M, Munguba H, Varemo L, et al. Transcriptional Convergence of Oligodendrocyte Lineage Progenitors during Development. Dev Cell 2018; 46:504-17.e7.
[14] Brandt R, Mascher M, Thiel J. Laser Capture Microdissection-Based RNA-Seq of Barley Grain Tissues. Methods Mol Biol 2018; 1723:397-409.
[15] Sarantopoulou D, Tang S Y, Ricciotti E, Lahens N F, Lekkas D, Schug J, et al. Comparative evaluation of RNA-Seq library preparation methods for strand-specificity and low input. Sci Rep 2019; 9:13477.
[16] Chao H P, Chen Y, Takata Y, Tomida M W, Lin K, Kirk J S, et al. Systematic evaluation of RNA-Seq preparation protocol performance. BMC Genomics 2019; 20:571.
[17] Godia M, Mayer F Q, Nafissi J, Castello A, Rodriguez-Gil J E, Sanchez A, et al. A technical assessment of the porcine ejaculated spermatozoa for a sperm-specific RNA-seq analysis. Syst Biol Reprod Med 2018; 64:291-303.
[18] Li F, Kaczor-Urbanowicz K E, Sun J, Majem B, Lo H C, Kim Y, et al. Characterization of Human Salivary Extracellular RNA by Next-generation Sequencing. Clin Chem 2018; 64:1085-95.
[19] Song B, Du J, Feng Y, Gao Y J, Zhao J S. Co-expressed differentially expressed genes and long non-coding RNAs involved in the celecoxib treatment of gastric cancer: An RNA sequencing analysis. Exp Ther Med 2016; 12:2455-68.
[20] Ramskold D, Luo S, Wang Y C, Li R, Deng Q, Faridani O R, et al. Full-length mRNA-Seq from single-cell levels of RNA and individual circulating tumor cells. Nat Biotechnol 2012; 30:777-82.
[21] Moll P, Ante M, Seitz A, Reda T. QuantSeq 3′ mRNA sequencing for RNA quantification. Nature Methods 2014; 11:i-iii.
[22] Ma F, Fuqua B K, Hasin Y, Yukhtman C, Vulpe C D, Lusis A J, et al. A comparison between whole transcript and 3′ RNA sequencing methods using Kapa and Lexogen library preparation methods. BMC Genomics 2019; 20:9.
[23] Han L, Wu H J, Zhu H, Kim K Y, Marjani S L, Riester M, et al. Bisulfite-independent analysis of CpG island methylation enables genome-scale stratification of single cells. Nucleic Acids Res 2017; 45:e77.
[24] Nakagawa H, Hasumi K, Woo J T, Nagai K, Wachi M. Generation of hydrogen peroxide primarily contributes to the induction of Fe(II)-dependent apoptosis in Jurkat cells by (−)-epigallocatechin gallate. Carcinogenesis 2004; 25:1567-74.
[25] Bolger A M, Lohse M, Usadel B. Trimmomatic: a flexible trimmer for Illumina sequence data. Bioinformatics 2014; 30:2114-20.
[26] Trapnell C, Roberts A, Goff L, Pertea G, Kim D, Kelley D R, et al. Differential gene and transcript expression analysis of RNA-seq experiments with TopHat and Cufflinks. Nat Protoc 2012; 7:562-78.
[27] Li H, Handsaker B, Wysoker A, Fennell T, Ruan J, Homer N, et al. The Sequence Alignment/Map format and SAMtools. Bioinformatics 2009; 25:2078-9.
[28] Liao Y, Smyth G K, Shi W. The Subread aligner: fast, accurate and scalable read mapping by seed-and-vote. Nucleic Acids Res 2013; 41:e108.
[29] Anders S, Pyl P T, Huber W. HTSeq—a Python framework to work with high-throughput sequencing data. Bioinformatics 2015; 31:166-9.
[30] Hashimshony T, Senderovich N, Avital G, Klochendler A, de Leeuw Y, Anavy L, et al. CEL-Seq2: sensitive highly-multiplexed single-cell RNA-Seq. Genome Biol 2016; 17:77.
[31] Zheng G X, Terry J M, Belgrader P, Ryvkin P, Bent Z W, Wilson R, et al. Massively parallel digital transcriptional profiling of single cells. Nat Commun 2017; 8:14049.
[32] Pan X, Weissman S M. An approach for global scanning of single nucleotide variations. Proc Natl Acad Sci USA 2002; 99:9346-51.
[33] De Dominici M, Porazzi P, Soliera A R, Mariani S A, Addya S, Fortina P, et al. Targeting CDK6 and BCL2 Exploits the “MYB Addiction” of Ph(+) Acute Lymphoblastic Leukemia. Cancer Res 2018; 78:1097-109.
[34] Erbilgin Y, Hatirnaz Ng O, Can I, Firtina S, Kucukcankurt F, Karaman S, et al. Prognostic evidence of LEF1 isoforms in childhood acute lymphoblastic leukemia. International journal of laboratory hematology 2021.
[35] Wu H B, Lv W F, Wang Y X, Li Y Y, Guo W. BCL6 promotes the methotrexate-resistance by upregulating ZEB1 expression in children with acute B lymphocytic leukemia. European review for medical and pharmacological sciences 2018; 22:5240-7.
[36] Steger B, Floro L, Amberger D C, Kroell T, Tischer J, Kolb H J, et al. WT1, PRAME, and PR3 mRNA Expression in Acute Myeloid Leukemia (AML). Journal of immunotherapy (Hagerstown, Md.: 1997) 2020; 43:204-15.
[37] Fukuda Y, Wang Y, Lian S, Lynch J, Nagai S, Fanshawe B, et al. Upregulated heme biosynthesis, an exploitable vulnerability in MYCN-driven leukemogenesis. JCI Insight 2017; 2.
[38] Ghetti M, Vannini I, Storlazzi C T, Martinelli G, Simonetti G. Linear and circular PVT1 in hematological malignancies and immune response: two faces of the same coin. Molecular cancer 2020; 19:69.
[39] Zhang H, Xu H B, Kurban E, Luo H W. LncRNA SNHG14 promotes hepatocellular carcinoma progression via H3K27 acetylation activated PABPC1 by PTEN signaling. Cell death & disease 2020; 11:646.
[40] Lin I Y, Yen C H, Liao Y J, Lin S E, Ma H P, Chan Y J, et al. Identification of FKBP11 as a biomarker for hepatocellular carcinoma. Anticancer Res 2013; 33:2763-9.
[41] Huang Z, Chu L, Liang J, Tan X, Wang Y, Wen J, et al. H19 Promotes HCC Bone Metastasis Through Reducing Osteoprotegerin Expression in a Protein Phosphatase 1 Catalytic Subunit Alpha/p38 Mitogen-Activated Protein Kinase-Dependent Manner and Sponging microRNA 200b-3p. Hepatology (Baltimore, Md.) 2020.
[42] Lin C C, Liu T W, Yeh M L, Tsai Y S, Tsai P C, Huang C F, et al. Significant down-regulation of growth hormone receptor expression revealed as a new unfavorable prognostic factor in hepatitis C virus-related hepatocellular carcinoma. Clinical and molecular hepatology 2021; 27:313-28.
[43] Liu Y, Zhu X, Zhu J, Liao S, Tang Q, Liu K, et al. Identification of differential expression of genes in hepatocellular carcinoma by suppression subtractive hybridization combined cDNA microarray. Oncol Rep 2007; 18:943-51.
[44] Zhao Y, Li M, Yao X, Fei Y, Lin Z, Li Z, et al. HCAR1/MCT1 Regulates Tumor Ferroptosis through the Lactate-Mediated AMPK-SCD1 Activity and Its Therapeutic Implications. Cell Rep 2020; 33:108487.
[45] Morrissy A S, Morin R D, Delaney A, Zeng T, McDonald H, Jones S, et al. Next-generation tag sequencing for cancer gene expression profiling. Genome Res 2009; 19:1825-35.
[46] Hashimshony T, Wagner F, Sher N, Yanai I. CEL-Seq: single-cell RNA-Seq by multiplexed linear amplification. Cell Rep 2012; 2:666-73.
[47] Lu C, Zhang J, He S, Wan C, Shan A, Wang Y, et al. Increased alpha-tubulinlb expression indicates poor prognosis and resistance to chemotherapy in hepatocellular carcinoma. Dig Dis Sci 2013; 58:2713-20.
[48] Ziegenhain C, Vieth B, Parekh S, Reinius B, Guillaumet-Adkins A, Smets M, et al. Comparative Analysis of Single-Cell RNA Sequencing Methods. Mol Cell 2017; 65:631-43.e4.
[49] Picelli S, Faridani O R, Bjorklund A K, Winberg G, Sagasser S, Sandberg R. Full-length RNA-seq from single cells using Smart-seq2. Nat Protoc 2014; 9:171-81.
[50] Donczew R, Hahn S. Mechanistic Differences in Transcription Initiation at TATA-Less and TATA-Containing Promoters. Mol Cell Biol 2018; 38.
[51] Di Giammartino D C, Nishida K, Manley J L. Mechanisms and consequences of alternative polyadenylation. Mol Cell 2011; 43:853-66.

What is claimed is:

1. A set of primers for performing multiplex RNA sequencing, comprising 4 sets of oligonucleotides, including:
(1) An RT primer for capturing an mRNA and synthesizing a 1^{st} strand of cDNA;
(2) A 2^{nd} strand cDNA synthesis primer for 2^{nd} strand cDNA synthesis starting from multiple sites along the 1^{st} strand of cDNA;
(3) A PCR primer 1 for capturing a 5′ end sequence of the RT primer so as to capture the poly-dA/dT ends of a cDNA during library amplification in combination with PCR primer 2; and
(4) A PCR primer 2 for capturing a 5′ end sequence of the 2^{nd} strand primer so as to capture the distal end of the poly-dA/dT end of a cDNA during library amplification in combination with PCR primer 1.

2. The set of primers of claim 1, wherein the RT primer comprises:
(1) an oligo-dT at a 3′ end of the RT primer, composed of multiple thymine (T), recorded as Tn, wherein n is the number of the T, with a length of 14-30 bases (bases are also known as nucleotides, and ‘base’ and ‘nucleotide’ are identical and used alternatively herein);
(2) a sample barcode with a length of 4-12 bases;
(3) a unique molecular identifier (UMI), comprising 4 repeats of NBB, where N is adenine (A), thymine (T), cytosine (C), or guanine (G) and B is T, C, or G;
(4) an adapter sequence, for anchoring a 5′ adapter corresponding to the specific sequencing platform.

3. The set of primers of claim 2, wherein the 3′ end of oligo-dT is configured to be TnVN-3′, TnV-3′, Tn-3′ or TnN-3′, where N is adenine(A), thymine(T), cytosine(C) or guanine(G), and V is C, A, or G, and n is the integer number of T, ranging from 6-11.

4. The set of primers of claim 1, wherein the UMI is oligo nucleotides of 1-12 bases in length, and sample barcode has length between 4-12 bases, and the index has length of 6 bases.

5. The set of primers of claim 4, wherein the bases of the primers, at any possible site, are random bases as any one kind of A, T, C or G; and/or semi-random bases as any three/two kind of A, T, C or G.

6. The set of primers of claim 1, wherein a random sequence or a semi-random sequence of bases are at a 3′ end of the 2^{nd} cDNA strand synthesis primer, and vary from 6-11 bases, or a designated order of sequences specifically capture the 2^{nd} strand of a given transcript; and the 5′ end of the primer for the 2^{nd}-strand cDNA synthesis primer is compatible with a 3′ adapter sequence, including a P7 attachment site, corresponding to a specific sequencing platform.

7. The set of primers of claim 1, wherein the set of primers contains PCR primers for amplification of cDNA comprising PCR primer 1 and PCR primer 2, and the PCR primers provide an anchoring tag for a sequencing primer to perform sequencing of a cDNA insert, and a molecular UMI, a sample barcode, and a batch index of samples, on a specific sequencing platform.

8. The set of primers of claim 1, wherein the 3′ end of the second cDNA strand synthesis primer comprises one to three B, where B is C, T, or G.

9. The set of primers of claim 2, wherein sequence of the RT primer is listed in SEQ ID NO. 1.

10. The set of primers of claim 1, wherein sequence of the second cDNA strand synthesis primer is listed in SEQ ID NO.2.

11. The set of primers of claim 7, wherein a sequence of the PCR primer 1 is listed in SEQ ID NO.3.

12. The set of primers of claim 7, wherein a sequence of the PCR primer 2 is listed in SEQ ID NO.4.

13. A method of multiplexable RNA sequencing using the set of primers of claim 1, comprising:
(1) obtaining one or more than one sample of RNA;
(2) reverse transcription of the RNA of step (1) through the RT primer, leading to selection of mRNA with a polyA tail for the synthesis of a first cDNA strand;
(3) removal of the mRNA from the first strand cDNA and all other RNAs;
(4) synthesis of one or multiple second strands of cDNA with the 2^{nd} strand cDNA synthesis primer initiating from a single or multiple sites along the 1^{st} strand of cDNA;
(5) pooling the products of samples (sample number greater or equal to 1, and less than or equal to 500) before or after the synthesis of the 2^{nd} strand of cDNA at the step (4), into one tube, followed by cDNA purification and concentration;
(6) PCR amplification using double or single stranded cDNA as a substrate, resulting in preliminary libraries that contain cDNA sequences corresponding to 3′ end regions of mRNAs, using the PCR primer 1 and the PCR primer 2;
(7) DNA size selection, DNA enrichment, DNA recovery or DNA purification are conducted based on the libraries obtained in step (6), resulting in an optimal size of cDNA that are suitable for sequencing on a specific sequencing platform;
(8) sequencing via NGS (next generation sequencing) platforms of libraries from step (7), resulting in transcriptomic data of pooled samples;
(9) Bioinformatical analysis of the data obtained in step (8), resulting in a transcriptome profile of each individual sample.

14. The method of claim 13, wherein the step (6) is followed by a second round of PCR that produces optimized libraries for a specific NGS platform; and a same pair of PCR primers are used during the second round of PCR; and a new set of primers partially compatible with the PCR primer 1 and the PCR Primer 2, and with an extended part of sequences on the 5′ end of the primers fitting the desired different sequencing platforms.

15. The method of claim 13, wherein the optimal size of cDNA, including an insert cDNA sequence and adapter sequences, in step (7) is a size range within 250 bp to 1000 bp.

16. The method of claim 13, wherein in a NGS sequencing platform in step (8), the sequencing strategy is either paired end or single end; paired end 150 bp sequencing, single end or paired end sequencing on varying length of libraries.

17. The method of claim 13, wherein the method is extended and modified for construction of library and sequencing of a set of specific corresponding mRNAs/genes, wherein the second strand synthesis primers in claim 1 step (2) are with a specific 3′ end sequence that each captures a 5′ end region of a target 1^{st} strand cDNAs.

18. The method of claim 13, wherein software is used for the bioinformatic analysis of the data obtained in step (8), and wherein the procedure of bioinformatical analysis in step (9) comprises:
(1) preliminary process of the transcriptome profile data from step (8), comprising one or more of: quality control, index demultiplex, barcode demultiplex, removal of polyA sequences, removal of sequencing adapters and low-quality bases, and elimination of reads amplification bias according to UMI;
(2) the analysis of the sequencing data processed in step (1) comprising one or more of: alignment, quality control of the data after mapping, calculation and normalization of reads count, calculation of detected genes, Pearon correlation coefficient evaluation; and analysis of Venn diagram, gene enrichment, GO enrichment, heatmap, and differential genes; wherein the information decoding analysis method of the sequencing data in the step (8) analyzes the termination site of the transcriptome of a specific research object.

19. The method of claim 13, wherein all or a part of the innovation, process or software are based to construct an instrument.

20. The method of claim 13, are used in biological science research, medical research, clinical diagnosis or drug development; and agricultural, plant, animal, and microbial research; and applications include development, tumors, immunity, genetic diseases, experimental targeting, viruses, animal husbandry, traditional Chinese medicine, and drug research and development.
